(19) United States
(12) Patent Application Publication
(10) Pub. No.: US 2024/0398697 A1
(43) Pub. Date: Dec. 5, 2024

(54) Injectable Formulations

(71) Applicant: The University of Liverpool, Liverpool, Merseyside (GB)

(72) Inventors: Steven Paul Rannard, Mickle Trafford, Chesire (GB); Andrew Owen, Liverpool, Merseyside (GB); Andrew Dwyer, Liverpool, Merseyside (GB); Catherine Unsworth, Liverpool, Merseyside (GB); James Hobson, Liverpool, Merseyside (GB); Paul Curley, Liverpool, Merseyside (GB); Joanne Sharp, Liverpool, Merseyside (GB); Ryan Donnelly, Belfast (GB); Yara NASER, Belfast (GB)

(21) Appl. No.: 18/696,258

(22) PCT Filed: Sep. 29, 2022
(86) PCT No.: PCT/GB2022/052475

§ 371 Date: Mar. 27, 2024

(30) Foreign Application Priority Data
Sep. 29, 2021 (GB) 2113944.9

(51) Int. Cl. A61K 9/00; A61K 31/454; A61K 31/498; A61K 45/06

(52) U.S. Cl. CPC A61K 9/0021; A61K 9/0024; A61K 31/454; A61K 31/498; A61K 45/06

(57) ABSTRACT

The present invention relates to solid compositions comprising microparticles of glecaprevir and/or pibrentasvir dispersed within a matrix comprising a first excipient and a second excipient. The present invention also relates to microneedle arrays, implantable rods, aqueous dispersions, and pharmaceutical compositions derived from said solid compositions and uses for the same.

### INTRODUCTION

The present invention relates to long acting injectable liquid formulations comprising an aqueous dispersion of microparticles of glecaprevir and/or pibrentasvir stabilised by a first excipient and a second excipient. The present invention also relates to solid compositions which may be used as a precursor for the long acting injectable formulations, aqueous dispersions, microneedle arrays, implantable rods, and pharmaceutical compositions, methods of producing such long acting injectable formulations, and medical uses for such formulations and methods of treatment using such formulations.

### BACKGROUND OF THE INVENTION

Hepatitis C virus (HCV) is a blood-borne virus spread predominantly through blood-to-blood contact, for example via contaminated blood products, the sharing of needles by intravenous drug users, and in clinical settings where sterilization is incorrectly implemented. It is classified into six genotypes, with different genotypes responding differently to treatments. HCV infection causes the disease Hepatitis C, which may present as acute or chronic, with the chronic condition eventually causing cirrhosis and liver cancer. Currently, no vaccine exists to protect against HCV infection, although a number of treatments have been developed that can achieve a sustained virological response in many patients.

Glecaprevir is an inhibitor of HCV non-structural protein 3/4A (NS3/NS4A) protease and its chemical structure is reproduced below.

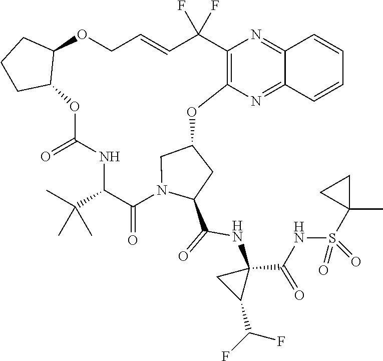 [SMILES: CC(C)(C)[C@@H]1NC(=O)O[C@@H]2CCC[C@H]2OC/C=C/C(F)(F)c2nc3ccccc3nc2O[C@@H]2C[C@@H](C(=O)N[C@]3(C(=O)NS(=O)(=O)C4(C)CC4)C[C@H]3C(F)F)N(C2)C1=O]

Pibrentasvir is an inhibitor of HCV non-structural protein 5A (NS5A) and its chemical structure is reproduced below.

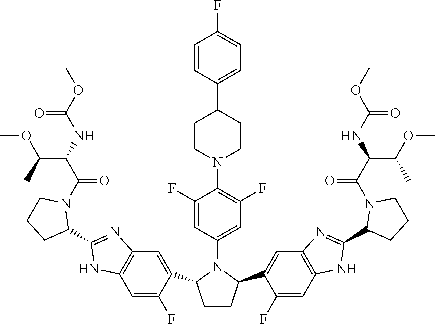

Glecaprevir and pibrentasvir are taken together as a combination therapy for the treatment of all six genotypes of HCV. The typical dosage regimen is 300/120 mg (glecaprevir/pibrentasvir) taken orally once daily, with the treatment duration being 8, 12 or 16 weeks depending on factors such as the genotype of the HCV infection, status of the patient and previous treatment(s) the patient may have received.

The lengthy nature of these oral dosing regimens means that there are issues with patient compliance, with patients either accidentally or consciously failing to follow their treatment plan. For example, patients may not take the medication prescribed, or may fail to attend follow-up appointments. This can cause the concentration of the drug to drop to ineffective levels for a period of time, lowering the effectiveness of the treatment and increasing the risk of relapse and onward transmission. There is, therefore, a need to provide HCV treatments that improve patient compliance and/or improve effectiveness of the treatment, for example, by removing the need to attend follow-up appointments.

### SUMMARY OF THE INVENTION

A first aspect of the present invention provides a solid composition comprising microparticles of glecaprevir and/or pibrentasvir dispersed within a matrix comprising a first excipient and a second excipient,
wherein the first excipient is selected from polyvinylpyrrolidone (PVP), polyvinyl alcohol (PVA), polyethylene glycol (PEG), hydroxypropyl methylcellulose (HPMC), polyoxyethylene-polyoxypropylene block copolymer (poloxamer), polyethoxylated castor oil, PVA-PEG graft copolymer, lactose, and sucrose, and
wherein the second excipient is selected from dioctyl sodium sulfosuccinate (AOT), benzalkonium chloride, sodium deoxycholate (NDC), poloxamer, Polysorbate 20, Polysorbate 80, sorbitan monolaurate, polyethylene glycol (15)-hydroxystearate, and vit-E-PEG succinate.

In an embodiment, the microparticles of glecaprevir and/or pibrentasvir are microparticles of pibrentasvir. Optionally, the first excipient is selected from polyethoxylated castor oil, PVP, lactose, sucrose, poloxamer, and PEG; and the second excipient is selected from benzalkonium chloride, AOT, poloxamer, Polysorbate 20, Polysorbate 80, and sorbitan monolaurate. Preferably, the first and second excipients are selected from the following combinations:
polyethoxylated castor oil and benzalkonium chloride
polyethoxylated castor oil and AOT
PVP and benzalkonium chloride
PVP and AOT
PVP and sorbitan monolaurate
lactose and benzalkonium chloride
lactose and AOT
lactose and poloxamer
sucrose and benzalkonium chloride
sucrose and AOT
sucrose and sorbitan monolaurate
poloxamer and benzalkonium chloride
poloxamer and AOT
poloxamer and Polysorbate 20
PEG and benzalkonium chloride
PEG and AOT
PEG and Polysorbate 20
PEG and sorbitan monolaurate

More preferably the first and second excipients are selected from the following combinations:
PVP and benzalkonium chloride
PVP and AOT
lactose and benzalkonium chloride
sucrose and AOT
poloxamer and benzalkonium chloride
poloxamer and Polysorbate 20
poloxamer and AOT
PEG and benzalkonium chloride.

In an alternative option, the first excipient is selected from PVA, PEG, HPMC, poloxamer, and PVA-PEG graft copolymer; and the second excipient is selected from AOT, Polysorbate 20, Polysorbate 80, NDC, polyethylene glycol (15)-hydroxystearate, and Vit-E-PEG succinate. Preferably, the first and second excipients are selected from the following combinations:
PVA and Polysorbate 20
PVA and Polysorbate 80
PVA and NDC
PVA and AOT
PVA and polyethylene glycol (15)-hydroxystearate
PVA and Vit-E-PEG succinate
PVA-PEG graft copolymer and AOT
PVA-PEG graft copolymer and NDC
Poloxamer and Polysorbate 20
Poloxamer and AOT
PEG and NDC
PEG and AOT
HPMC and NDC
HPMC and AOT
HPMC and Polysorbate 20
HPMC and Polysorbate 80
HPMC and Vit-E-PEG succinate
HPMC and polyethylene glycol (15)-hydroxystearate

More preferably the first and second excipients are selected from the following combinations:
PVA and Polysorbate 20
PVA and Polysorbate 80
PVA and NDC
PVA and AOT
PVA and polyethylene glycol (15)-hydroxystearate
PVA and Vit-E-PEG succinate
HPMC and Polysorbate 20
HPMC and Polysorbate 80
HPMC and Vit-E-PEG succinate
HPMC and polyethylene glycol (15)-hydroxystearate
HPMC and NDC
HPMC and AOT

In another embodiment, the microparticles of glecaprevir and/or pibrentasvir are microparticles of glecaprevir. Optionally, the first excipient is selected from polyethoxylated castor oil, PVP, lactose, sucrose, poloxamer, and PEG; and the second excipient is selected from benzalkonium chloride, AOT, poloxamer, Polysorbate 20, Polysorbate 80, and sorbitan monolaurate. Preferably, the first and second excipients are selected from the following combinations:
PVP and AOT
PVP and poloxamer
PVP and Polysorbate 20
PVP and Polysorbate 80
PVP and sorbitan monolaurate
lactose and AOT
sucrose and AOT
sucrose and poloxamer
sucrose and Polysorbate 20
sucrose and Polysorbate 80
   more preferably the first and second excipients are selected from the following combinations:
PVP and AOT
PVP and Polysorbate 20
PVP and Polysorbate 80

In an alternative option, the first excipient is selected from PVA, PEG, HPMC, poloxamer, and PVA-PEG graft copolymer; and the second excipient is selected from AOT, Polysorbate 20, Polysorbate 80, NDC, polyethylene glycol (15)-hydroxystearate, and Vit-E-PEG succinate. Preferably, the first and second excipients are selected from the following combinations:
PVA and AOT
PVA and Polysorbate 20
PVA and Polysorbate 80
PVA and NDC
PVA and polyethylene glycol (15)-hydroxystearate
PVA and Vit-E-PEG succinate
PVA-PEG graft copolymer and AOT
PVA-PEG graft copolymer and polyethylene glycol (15)-hydroxystearate
PVA-PEG graft copolymer and Polysorbate 80
poloxamer and NDC
HPMC and AOT
HPMC and NDC
HPMC and Polysorbate 80
HPMC and Vit-E-PEG succinate

More preferably the first and second excipients are selected from the following combinations:
PVA and AOT
PVA and Polysorbate 20
PVA and Polysorbate 80
PVA and NDC
PVA and polyethylene glycol (15)-hydroxystearate
PVA and Vit-E-PEG succinate
PVA-PEG graft copolymer and AOT
PVA-PEG graft copolymer and polyethylene glycol (15)-hydroxystearate
PVA-PEG graft copolymer and Polysorbate 80

In another embodiment, the microparticles of glecaprevir and/or pibrentasvir are microparticles of glecaprevir and pibrentasvir. Optionally, the first excipient is selected from polyethoxylated castor oil, PVP, lactose, sucrose, and poloxamer; and the second excipient is selected from benzalkonium chloride, AOT, poloxamer, Polysorbate 20, Polysorbate 80, and sorbitan monolaurate. Preferably, the first and second excipients are selected from the following combinations:
PVP and benzalkonium chloride
PVP and AOT
PVP and sorbitan monolaurate
PVP and Polysorbate 20
PVP and Polysorbate 80
lactose and AOT
sucrose and AOT
poloxamer and benzalkonium chloride
poloxamer and AOT
PVA and Polysorbate 20
PVA and Polysorbate 80
PVA and NDC
PVA and polyethylene glycol (15)-hydroxystearate
PVA and Vit-E-PEG succinate
PVA-PEG graft copolymer and AOT
HPMC and AOT
HPMC and polyethylene glycol (15)-hydroxystearate
HPMC and Polysorbate 80
HPMC and Vit-E-PEG succinate

Preferred combinations of first and second excipients for microparticles of glecaprevir and/or pibrentasvir are where the first excipient is PVP and the second excipient is AOT, Polysorbate 20, or Polysorbate 80, more preferably the second excipient is AOT.

The solid composition may comprise:
40 to 80 wt % glecaprevir and/or pibrentasvir combined;
5 to 50 wt % of the first excipient; and
1 to 30 wt % of the second excipient.

The solid composition may comprise:
50 to 70 wt % glecaprevir and/or pibrentasvir combined;
20 to 40 wt % of the first excipient; and
5 to 15 wt % of the second excipient.

The solid composition may comprise:
about 60 wt % glecaprevir and/or pibrentasvir combined;
about 30 wt % of the first excipient; and
about 10 wt % of the second excipient.

The microparticles may have a particle diameter in the range of 10 to 2500 nm, preferably between 20 nm and 2000 nm, more preferably between 50 nm and 1500 nm, further preferably between 100 nm and 1000 nm, and most preferably between 150 and 450 nm.

The microparticles may have a polydispersity less than or equal to 0.8, preferably less than or equal to 0.6, more preferably less than or equal to 0.5.

A second aspect of the present invention provides a process for preparing a solid composition according to the first aspect of the present invention, the process comprising:
(a) preparing an oil-in-water emulsion comprising:
   an oil phase comprising glecaprevir and/or pibrentasvir; and
   an aqueous phase comprising a first and second excipient, each as defined in any embodiment of the first aspect of the present invention; and
(b) removing the oil and water from the oil-in-water emulsion to form the solid composition.

The step of removing the oil and water from the oil-in-water emulsion may comprise spray drying or freeze-drying.

A third aspect of the present invention relates to an aqueous dispersion comprising a plurality of microparticles of glecaprevir and/or pibrentasvir dispersed in an aqueous medium and stabilised by a mixture of a first excipient and a second excipient;
wherein the first excipient is selected from polyvinylpyrrolidone (PVP), polyvinyl alcohol (PVA), polyethylene glycol (PEG), hydroxypropyl methylcellulose (HPMC), polyoxyethylene-polyoxypropylene block copolymer (poloxamer), polyethoxylated castor oil, PVA-PEG graft copolymer, lactose, and sucrose, and
wherein the second excipient is selected from sodium sulfosuccinate (AOT), benzalkonium chloride, dioctyl sodium deoxycholate (NDC), poloxamer, Polysorbate 20, Polysorbate 80, sorbitan monolaurate, polyethylene glycol (15)-hydroxystearate, and vit-E-PEG succinate.

The aqueous dispersion may comprise microparticles of glecaprevir and/or pibrentasvir, first excipients, and second excipients as defined in any embodiment of the first aspect of the present invention in the aqueous medium.

The glecaprevir and/or pibrentasvir may be present in the aqueous dispersion at a concentration of 100 to 1000 mg/mL, preferably 200 to 900 mg/mL, more preferably 300 to 800 mg/mL, and most preferably 500 to 700 mg/mL.

A fourth aspect of the present invention relates to a process for preparing an aqueous dispersion according to the third aspect of the present invention, the process comprising dispersing a solid composition according to the first aspect of the present invention in an aqueous medium.

A fifth aspect of the present invention relates to a pharmaceutical composition comprising the solid composition of the first aspect of the present invention, or the aqueous dispersion of the third aspect of the present invention and, optionally, one or more further pharmaceutically acceptable excipients.

A sixth aspect of the present invention relates to an injectable formulation comprising the solid composition of the first aspect of the present invention, or the aqueous dispersion of the third aspect of the present invention, or the pharmaceutical composition of the fifth aspect of the present invention.

The injectable formulation may be a subcutaneously or intramuscularly injectable formulation, optionally wherein the injectable formulation is suitable for provision in depot form.

A seventh aspect of the present invention relates to a method of producing an implantable rod comprising the steps of compressing a solid composition according to the first aspect of the present invention and heating the compressed solid composition for a period of time.

The solid composition may be compressed in a mould, optionally the mould being cylindrical in form.

The solid composition may be heated to a temperature from 60 to 160° C., preferably from 80 to 140° C., more preferably from 100 to 120° C., most preferably about 110° C.

The compression may occur under a reduced pressure atmosphere.

The heating step may take place for a period of from 1 minute to 30 minutes, preferably from 2 minutes to 25 minutes, more preferably from 5 minutes to 15 minutes, most preferably about 10 minutes.

The method may further comprise a step of cooling the rod, optionally the cooling taking place under a reduced pressure atmosphere.

It will be understood that other implant manufacturing processes that are known to those skilled in the art (such as injection moulding) may also be used to produce the implantable rods.

An eighth aspect of the present invention relates to an implantable rod formed by the first method of the present invention.

A ninth aspect of the present invention relates to an implantable rod comprising microparticles of glecaprevir and/or pibrentasvir dispersed within a monolith comprising a first excipient and a second excipient,
wherein the first excipient is selected from polyvinylpyrrolidone (PVP), polyvinyl alcohol (PVA), polyethylene glycol (PEG), hydroxypropyl methylcellulose (HPMC), polyoxyethylene-polyoxypropylene block copolymer (poloxamer), polyethoxylated castor oil, PVA-PEG graft copolymer, lactose, and sucrose, and
   wherein the second excipient is selected from dioctyl sodium sulfosuccinate (AOT), benzalkonium chloride, sodium deoxycholate (NDC), poloxamer, Polysorbate 20, Polysorbate 80, sorbitan monolaurate, polyethylene glycol (15)-hydroxystearate, and vit-E-PEG succinate.

The microparticles glecaprevir and/or pibrentasvir, first excipient, and/or second excipient of the implantable rods of the eleventh aspect of the present invention may be as defined in the first aspect of the present invention.

A tenth aspect of the present invention relates to a method of producing a microneedle array comprising microneedles of a first composition arrayed on one face of a baseplate of a second composition, the method comprising the steps of:
a) dispersing a solid composition according to the first aspect of the present invention and at least one structural polymer in a solvent to form a microneedle precursor dispersion;
b) placing the microneedle precursor dispersion into a mould;
c) compressing the microneedle precursor dispersion in the mould and then drying to form microneedles comprising the first composition;
d) adding a baseplate precursor solution into the mould;
e) compressing the baseplate precursor solution and then drying to form the baseplate of the second composition; and
f) releasing the microneedle array from the mould.

Steps b) and c) may be repeated prior to steps d) to f).

The solvent may be an aqueous solvent, such as water.

The at least one structural polymer may be selected from PVA, PVP, and combinations thereof.

The baseplate precursor solution may comprise a base polymer selected from PVP and, optionally, one or more additives such as glycerol, dispersed in an aqueous solvent, such as water.

An eleventh aspect of the present invention relates to a microneedle array produced by the method of the twelfth aspect of the present invention.

A twelfth aspect of the present invention relates to a microneedle array comprising microneedles of a first composition arrayed on one face of a baseplate of a second composition,
wherein the first composition comprises microparticles of glecaprevir and/or pibrentasvir dispersed within a monolith comprising:
   a first excipient selected from polyvinylpyrrolidone (PVP), polyvinyl alcohol (PVA), polyethylene glycol (PEG), hydroxypropyl methylcellulose (HPMC), polyoxyethylene-polyoxypropylene block copolymer (poloxamer), polyethoxylated castor oil, PVA-PEG graft copolymer, lactose, and sucrose,
   a second excipient selected from dioctyl sodium sulfosuccinate (AOT), benzalkonium chloride, sodium deoxycholate (NDC), poloxamer, Polysorbate 20, Polysorbate 80, sorbitan monolaurate, polyethylene glycol (15)-hydroxystearate, and vit-E-PEG succinate, and
   at least one structural polymer.

The microparticles of glecaprevir and/or pibrentasvir, first excipient, and/or second excipient are as defined in the first aspect of the present invention.

The at least one structural polymer may be selected from PVA, PVP, and combinations thereof.

The second composition may comprise a base polymer, such as PVP, and, optionally, one or more additives, such as glycerol.

A thirteenth aspect of the present invention relates to a solid composition according to the first aspect of the present invention, an aqueous dispersion according to the third aspect of the present invention, a pharmaceutical composition according to the fifth aspect of the present invention, an injectable formulation according to the sixth aspect of the present invention, an implantable rod according to the eighth or ninth aspects of the present invention, or a microneedle array according to the eleventh and twelfth aspects of the present invention, for use as a medicament.

A fourteenth aspect of the present invention relates to a solid composition according to the first aspect of the present invention, an aqueous dispersion according to the third aspect of the present invention, a pharmaceutical composition according to the fifth aspect of the present invention, an injectable formulation according to the sixth aspect of the present invention, an implantable rod according to the eighth or ninth aspects of the present invention, or a microneedle array according to the eleventh and twelfth aspects of the present invention, for use in the treatment and/or prevention of HCV infection and/or Hepatitis C.

An aqueous dispersion, a pharmaceutical composition, or an injectable formulation for use according to the fourteenth aspect of the present invention, wherein the concentration of glecaprevir and/or pibrentasvir is 100 to 1000 mg/mL, preferably 200 to 900 mg/mL, more preferably 300 to 800 mg/mL, and most preferably 500 to 700 mg/mL.

An implantable rod for use according to the fourteenth aspect of the present invention, wherein the concentration of glecaprevir and/or pibrentasvir in the implantable rod is in the range of 40 to 80 wt %, preferably 50 to 70 wt %, most preferably about 60 wt %.

A microneedle array for use for use according to the fourteenth aspect of the present invention, wherein the microneedle array contains a mass of glecaprevir and/or pibrentasvir in the range of between 1 and 20 mg of glecaprevir and/or pibrentasvir, preferably between 2 and 10 mg, more preferably about 5 mg.

A fifteenth aspect of the present invention relates to a method of treating and/or preventing HCV infection and/or Hepatitis C, the method comprising administering a therapeutically effective amount of a solid composition according to the first aspect of the present invention, an aqueous dispersion according to the third aspect of the present invention, a pharmaceutical composition according to the fifth aspect of the present invention, an injectable formulation according to the sixth aspect of the present invention, an implantable rod according to the eighth or ninth aspects of the present invention, or a microneedle array according to the eleventh and twelfth aspects of the present invention to a patient suffering from or at risk of suffering from HCV infection and/or Hepatitis C.

The concentration of glecaprevir and/or pibrentasvir within the aqueous dispersion, the pharmaceutical composition, or the injectable formulation used in the method of the fifteenth aspect of the present invention may 100 to 1000 mg/mL, preferably 200 to 900 mg/mL, more preferably 300 to 800 mg/mL, and most preferably 500 to 700 mg/mL.

The concentration of glecaprevir and/or pibrentasvir in the implantable rod may be in the range of 40 to 80 wt %, preferably 50 to 70 wt %, most preferably about 60 wt %.

The microneedle array may contain a mass of glecaprevir and/or pibrentasvir in the range of between 1 and 20 mg of glecaprevir and/or pibrentasvir, preferably between 2 and 10 mg, more preferably about 5 mg.

The aqueous dispersion, the pharmaceutical composition, the injectable formulation, or the implantable rod may form a depot within the body of the patient, optionally wherein the depot maintains a therapeutically effective concentration of glecaprevir and/or pibrentasvir within the body of the patient for a period of at least two weeks, preferably at least three weeks, more preferably at least one month, and most preferably at least two months.

The microneedle array may gradually release glecaprevir and/or pibrentasvir, optionally wherein the microneedle array maintains a therapeutically effective concentration of glecaprevir and/or pibrentasvir within the body of the patient for a period of at least 4 hours, preferably at least 6 hours, more preferably at least 12 hours, and most preferably at least 24 hours.

The patient may require dosing with the aqueous dispersion, the pharmaceutical composition, the injectable formulation, or the implantable rod up to three times, preferably up to two times, most preferably only once, to maintain a therapeutically effective concentration of glecaprevir and/or pibrentasvir for the duration of the treatment.

The patient may require dosing of the microneedle array up to six times per day, preferably up to four times per day, more preferably twice a day, and most preferably once a day, to maintain a therapeutically effective concentration of glecaprevir and/or pibrentasvir in the patient for the duration of the treatment.

### BRIEF DESCRIPTION OF THE DRAWINGS

FIG. 1 shows a summary of the dispersions formed from the pibrentasvir solid compositions formed from DCM-based emulsions by emulsion templated freeze drying as described in Example 1.

FIG. 2 shows a summary of the dispersions formed from the pibrentasvir solid compositions formed from DCM-based emulsions by emulsion templated freeze drying as described in Example 2.

FIG. 3 shows a summary of the dispersions formed from the pibrentasvir solid compositions formed from ethyl acetate based emulsions by emulsion templated freeze drying as described in Example 3.

FIG. 4 shows a summary of the dispersions formed from the pibrentasvir solid compositions formed from ethyl acetate based emulsions by emulsion templated freeze drying as described in Example 4.

FIG. 5 shows a summary of the dispersions formed from the glecaprevir solid compositions formed from DCM-based emulsions by emulsion templated freeze drying as described in Example 6.

FIG. 6 shows a summary of the dispersions formed from the glecaprevir solid compositions formed from ethyl acetate based emulsions by emulsion templated freeze drying as described in Example 7.

FIG. 7 shows a summary of the dispersions formed from the glecaprevir solid compositions formed from DCM-based emulsions by emulsion templated freeze drying as described in Example8.

FIGS. 8A and 8B show the blood plasma concentrations of Glecaprevir and Pibrentasvir respectively over time following intramuscular injection of an aqueous dispersion of the glecaprevir₃₀/pibrentasvir₃₀/Plasdone C15₃₀/AOT₁₀ formulation in male Sprague Dawley rats, as set out in Example 14. Each group received injections of varying size and constant concentration (thereby varying the total drug mass administered), with the highest blood plasma concentrations found in the largest injections and the lowest blood plasma concentrations found in the smallest injections. For both glecaprevir and pibrentasvir, Group 1 maintained the highest concentration, Group 2 maintained the second highest concentration, and Group 3 maintained the lowest concentration.

FIGS. 9A and 9B show the blood plasma concentrations of Glecaprevir and Pibrentasvir respectively over time following intramuscular injection of an aqueous dispersion of the glecaprevir₃₀/pibrentasvir₃₀/Plasdone C15₃₀/AOT₁₀ formulation in male Sprague Dawley rats, as set out in Example 15. Each group received injections of constant size and varying concentration (thereby varying the total drug mass administered). The highest blood plasma concentrations found in the highest dispersion concentrations and the lowest blood plasma concentrations found in the lowest dispersion concentrations. For both glecaprevir and pibrentasvir, Group 1 maintained the highest concentration, Group 2 maintained the second highest concentration, and Group 3 maintained the lowest concentration.

FIGS. 10A and 10B show the blood plasma concentrations of Glecaprevir and Pibrentasvir respectively over time following subcutaneous implantation in male Sprague Dawley rats of rods formed from the glecaprevir₃₀/pibrentasvir₃₀/Plasdone C15₃₀/AOT₁₀ formulation, as set out in Example 16.

FIGS. 11A and 11B show the blood plasma concentrations of Glecaprevir and Pibrentasvir respectively over time following intramuscular injection of an aqueous dispersion of one of a glecaprevir₆₀/Plasdone C15₃₀/AOT₁₀ formulation, a pibrentasvir₆₀/Plasdone C15₃₀/AOT₁₀ formulation, or a glecaprevir₃₀/pibrentasvir₃₀/Plasdone C15₃₀/AOT₁₀, formulation in male Sprague Dawley rats, as set out in Example 17. For glecaprevir in FIG. 11A, Group 3 showed the quickest decrease in concentration, followed by Group 1, and then Group 4. For pibrentasvir in FIG. 11B, all Groups showed similar concentrations over time.

FIGS. 12A and 12B show the ratio of liver concentration to blood plasma concentration for the intramuscularly administered compositions after 91-days compared to the same ratio for orally administered Maviret, as set out in Example 18.

FIG. 13 shows plan (left) and profile (right) views of a typical microneedle array produced in the method of Example 19.

FIG. 14 shows the results of the microneedle insertion study of Example 20. A shows the Insertion % for the microneedle arrays, where B shows the first through fourth layers of Parafilm® M punctured by a typical array.

FIG. 15 shows the results of the microneedle compression testing performed in Example 20. A shows a typical microneedle array prior to compression. B shows the same microneedle array after compression. C shows the average height reduction % for the singly and doubly cast microneedle arrays.

FIG. 16 shows the results of the ex vivo skin deposition experiment performed in Example 21. A shows the cumulative mass of each of glecaprevir and pibrentasvir that diffused into the receiver compartment over 24 hours. B shows the total quantity of each of glecaprevir and pibrentasvir that is deposited into the skin after 24 hours. C shows the total quantity of each of glecaprevir and pibrentasvir delivered (i.e. into the receiver compartment and the skin) after 24 hours.

FIGS. 17A and 17B show the blood plasma concentration of glecaprevir and pibrentasvir respectively following intravenous (IV—5 mg/kg dose of each drug), intramuscular (IM—37.5 mg of solid composition dispersed in 300 μL of water), and microneedle (MN—8 mg of each drug) administration in the experiment of Example 22. For glecaprevir in FIG. 17A, the IV concentration reduced rapidly, the MN concentration was maintained for longer, and the IM concentration was maintained for the longest. For pibrentasvir in FIG. 17B, the IV concentration reduced rapidly, the MN concentration was maintained for longer, and the IM concentration was maintained for the longest.

### DEFINITIONS

The term “viral infection” is used herein to refer to viral infections in general, including by HCV. Although the initial investigation is in the context of HCV, it will be understood that glecaprevir and pibrentasvir may also have broader antiviral activity.

Microparticles are, for the purposes of the present invention, considered to be any particle with a Z-average hydrodynamic diameter (as measured using dynamic light scattering—DLS) below 5 μm. This includes particles with sizes ranging from 1 to 1000 nm, commonly defined as nanoparticles.

For the avoidance of doubt, it is noted that “microparticles of glecaprevir and/or pibrentasvir” may refer to microparticles of glecaprevir and pibrentasvir wherein each microparticle may comprise both glecaprevir and pibrentasvir, wherein each microparticle may comprise one of glecaprevir and pibrentasvir, or wherein some microparticles comprise both glecaprevir and pibrentasvir and other microparticles comprise one of glecaprevir and pibrentasvir. Alternatively, microparticles of “glecaprevir and/or pibrentasvir” may refer to microparticles of glecaprevir or microparticles of pibrentasvir.

Unless otherwise stated, the term polydispersity index (Pdl), is in reference to the measurement provided by dynamic light scattering, in which perfect monodispersity is 0.

The term “consisting essentially of” is used herein to denote that a given product or method consists of only designated materials or steps and optionally other materials or steps that do not materially affect the characteristic(s) of the claimed invention. Suitably, a product which consists essentially of a designated material (or materials) comprises greater than or equal to 85% of the designated material, more suitably greater than or equal to 90%, more suitably greater than or equal to 95%, most suitably greater than or equal to 98% of the designated material(s).

Unless otherwise stated, the weight percentages (“wt %”) discussed herein relate to the % by weight of a particular constituent as a proportion of the total weight of the composition.

Unless otherwise stated, the weight/volume percentages (“w/v %”) discussed herein relate to the weight of the indicated material (in grams) per 100 mL of solvent.

It is to be appreciated that references to “preventing” or “prevention” relate to prophylactic treatment and includes preventing, limiting or delaying a viral infection following a patient's exposure to a virus. This may involve preventing, limiting or delaying the appearance of clinical symptoms developing in a patient that may be afflicted with or exposed to the virus but does not yet experience or display clinical or subclinical symptoms of the state, disorder or condition. Such prevention may prevent or reduce onward transmission of the virus.

It will be further appreciated that references to “treatment” or “treating” of HCV infection and/or Hepatitis C includes: (1) inhibiting the symptoms of the infection, i.e., arresting, reducing or delaying the development of the disease or a relapse thereof (in case of maintenance treatment) or at least one clinical or subclinical symptom thereof; or (2) relieving or attenuating the infection, i.e. causing regression of the state, disorder or condition or at least one of its clinical or subclinical symptoms. Such treatment may prevent or reduce onward transmission of HCV.

In the context of the invention, the terms “preventing” or “prevention” should not be considered to refer only to formulations which are completely effective in treating an infection, but also to cover formulations which are partially effective as well.

Moreover, when considered from the perspective of a population of patients for treatment, the terms “preventing” and “prevention” should be considered to cover formulations which are useful at reducing the rate of incidence of HCV infection in that target population, as well as medicaments which are useful at completely eradicating the HCV infection from that target population.

A “therapeutically effective amount” means the amount of pharmaceutically active compound that, when administered to a patient for treating and/or preventing a disease, is sufficient to effect such treatment/prevention for the HCV infection. The “therapeutically effective amount” will vary depending on the mass ratio of glecaprevir and pibrentasvir, the formulation and route of administration, the severity of the infection and the age, weight, etc., of the patient to be treated.

Syringeability is a measure of whether a solution, dispersion, or suspension is suitable for administration via injection. A composition is considered to be syringable if it can be manually passed through a 25G needle. It will be understood that the combination of the composition and 25G needle is purely to establish that a given composition is syringable and that the compositions may be used in combination with needles of a lower gauge in practice.

### DETAILED DESCRIPTION

### Solid Compositions of Glecaprevir and/or Pibrentasvir

The first aspect of the present invention provides a solid composition comprising microparticles of glecaprevir and/or pibrentasvir dispersed within a solid excipient mixture comprising the first and second excipients.

The microparticles of glecaprevir and/or pibrentasvir may be microparticles of glecaprevir in the absence of pibrentasvir. Alternatively, the microparticles of glecaprevir and/or pibrentasvir may be microparticles of pibrentasvir in the absence of glecaprevir.

The microparticles of glecaprevir and/or pibrentasvir may be microparticles of glecaprevir and/or microparticles of pibrentasvir. In other words, the solid composition comprises microparticles consisting of, or consisting essentially of, glecaprevir and/or microparticles consisting of, or consisting essentially of, pibrentasvir.

Alternatively, microparticles of glecaprevir and/or pibrentasvir may be microparticles of glecaprevir and pibrentasvir, wherein the microparticles comprise both glecaprevir and pibrentasvir.

In certain embodiments, the solid composition may comprise a mixture of the above microparticles. In other words, the solid composition may comprise at least two of microparticles of glecaprevir, microparticles of pibrentasvir, and microparticles of both glecaprevir and pibrentasvir.

The glecaprevir and/or pibrentasvir which comprises the microparticles may be amorphous (i.e. substantially non-crystalline in nature).

The solid excipient mixture is in the form of a matrix, which is highly porous in nature and rapidly dissolves on contact with aqueous solutions.

The solid composition of the present invention may be administered as it is to a patient, or further formulated to provide a pharmaceutical composition in the form of, for example, a tablet, capsule, lozenge, or a dispersible powder or granule formulation. In one embodiment, they may be formulated into an implantable rod.

The microparticles of the present invention have an average particle diameter of less than 5 micron (um). In a particular embodiment, the microparticles have an average particle diameter of between 10 nm and 2500 nm, preferably between 20 nm and 2000 nm, more preferably between 50 nm and 1500 nm, further preferably between 100 nm and 1000 nm, and most preferably between 100 and 500 nm. In embodiments, the microparticles are nanoparticles (i.e. they have a particle diameter in the range of 1 to 1000 nm). It will be understood that references to particle diameter are references to the Z-average hydrodynamic diameter of the microparticles.

The microparticles of the present invention may have a polydispersity less than or equal to 0.8, preferably less than or equal to 0.6, more preferably less than or equal to 0.5.

The particle diameter and polydispersity of the microparticles may be assessed by any suitable technique known in the art (e.g. laser diffraction, laser scattering, electron microscopy). In an embodiment of the invention, particle diameter (i.e. Z-average hydrodynamic diameter) is assessed by dispersing the solid composition in an aqueous medium and determining the particle diameter with a Malvern Panalytical Limited Zetasizer Ultra.

The solid composition may comprise particles or granules of larger size, for example, 5 to 30 microns (μm) in size, but each particle or granule may contain a plurality of microparticles of glecaprevir and/or pibrentasvir dispersed within a mixture of the first and second excipient. Alternatively, the solid composition may comprise a larger monolith, of any suitable shape or dimension. Furthermore, these monoliths, larger particles or granules disperse when the solid composition is mixed with an aqueous medium to form discrete microparticles of glecaprevir and/or pibrentasvir.

In an embodiment, the solid composition comprises a first excipient and a second excipient. In an alternative embodiment, the solid composition comprises further excipients selected from those listed herein as suitable first and/or second excipients.

### First Excipient

The first excipient is either a hydrophilic polymer or a sugar. In principle, any hydrophilic polymer or sugar suitable for use in pharmaceutical formulations may be employed in the present invention.

Particularly suitable polymers include: polyvinylpyrrolidones (including PVP k30, such as is available as Kollidon™ 30, PVP k17, such as is available as Kollidon™ 17 PF, PVP k15, such as is available as Plasdone™ C-15, and PVP k12, such as is available as Kollidon™ 12 PF); polyvinyl alcohol (PVA); hydroxypropylmethylcellulose (HPMC); polyethylene glycols (such as PEG 400, PEG 1000, and PEG 4000); polyvinyl alcohol-polyethylene glycol graft copolymers (such as those available in the Kollicoat™ range); non-ionic triblock copolymers composed of a central hydrophobic chain of polyoxypropylene (poly (propylene oxide)) flanked by two hydrophilic chains of polyoxyethylene (poly (ethylene oxide)), which are also known as poloxamers (such as those available in the Synperionics™, Pluronic™ and Kolliphor™ ranges, including Pluronic™ F68 and Pluronic™ F127); and polyethoxylated castor oil (such as Kolliphor™ EL).

It shall be appreciated that any weight average molecular weight (Mw) or number average molecular weight (Mn) values quoted herein may be determined by any suitable method known in the art for the particular polymer.

In embodiments, the polyvinyl alcohol has a weight average molecular weight (i.e. Mw) between 5000 and 200000 g/mol, suitably with a 75-90% hydrolysis level (i.e. % free hydroxyls). In a particular embodiment, the polyvinyl alcohol has a 75-90% hydrolysis level. In another embodiment, the polyvinyl alcohol has a 75-85% hydrolysis level. In a particular embodiment, the polyvinyl alcohol has a weight average molecular weight between 9000 and 10000 g/mol, suitably with an 80% hydrolysis level.

In embodiments, the polyvinylpyrrolidone has a weight average molecular weight of 1000 to 1,000,000 g/mol. In a particular embodiment, the polyvinylpyrrolidone has a weight average molecular weight of 1000 to 40000 g/mol, preferably 2000 to 20000 g/mol. In embodiments, the polyvinylpyrrolidone has a K value between 5 and 30, preferably between 10 and 20, most preferably between 12 and 17. Alternatively the K value may be about 12, about 15, or about 17. As is known in the art, K value is derived from relative viscosity measurements and calculated according to Fikentscher's equation.

A “poloxamer” is a non-ionic triblock copolymer comprising a central hydrophobic chain of polyoxypropylene, and hydrophilic chains of polyoxyethylene either side of this central hydrophobic chain. A “poloxamer” is typically named with the letter “P” followed by three numerical digits (e.g. P407), where the first two digits multiplied by 100 gives the approximate molecular mass of the polyoxypropylene chain, and the third digit multiplied by 10 provides the percentage polyoxyethylene content of the poloxamer. For example, P407 is a poloxamer having a polyoxypropylene molecular mass of about 4,000 g/mol and a polyoxyethylene content of about 70%, while P188 is a poloxamer having a polyoxypropylene molecular mass of about 1,800 g/mol and a polyoxyethylene content of about 80%. Poloxamers are also known as Pluronics®, as well as by several other commercial names. The poloxamer is suitably a pharmaceutically acceptable poloxamer. In a particular embodiment, the poloxamer is P407 or P188.

In an embodiment, the hydrophilic polymer is an HPMC. In a particular embodiment, the HPMC has a weight average molecular weight of 10000 to 400000 g/mol. In a particular embodiment, the HPMC has a weight average molecular weight of about 10000 g/mol.

Generally, monosaccharides, disaccharides, and oligosaccharides may be suitable in the solid composition of the present invention. Disaccharides are defined as carbohydrates consisting of two monosaccharide residues. Oligosaccharides are defined herein as carbohydrates consisting of between 3 and 10 monosaccharide residues.

Monosaccharides may be selected from ribose, arabinose, xylose, lyxose, ribulose, xylulose, allose, altrose, glucose, mannose, gulose, idose, galactose, talose, psicose, fructose, sorbose, and tagatose. Either of the D-or L-isomers may be used, with the naturally occurring isomer being preferred.

Disaccharides may be selected from any binary combination of the above monosaccharides. Preferred disaccharides are lactose and sucrose.

Oligosaccharides may be selected from any combination of the above monosaccharides.

In the present invention, the first excipient is selected from those hydrophilic polymers and sugars that are capable of stabilising microparticles of glecaprevir and/or pibrentasvir in an aqueous dispersion together with a second excipient as defined herein, and which are also suitable for pharmaceutical use (e.g. they are on the US Food and Drug Administration's Center for Drug Evaluation and Research (FDA CDER) list of inactive ingredients, especially those indicated as suitable for intramuscular injection).

### Second Excipient

The second excipient is a surfactant. In principle, any surfactant suitable for use in pharmaceutical formulations may be employed in the present invention. Examples of such surfactants include:
(a) non-ionic surfactants (e.g. ethoxylated triglycerides; fatty alcohol ethoxylates; alkylphenol ethoxylates; fatty acid ethoxylates; fatty amide ethoxylates; fatty amine ethoxylates; sorbitan alkanoates; ethylated sorbitan alkanoates; alkyl ethoxylates; Pluronics™; alkyl polyglucosides; stearol ethoxylates; alkyl polyglycosides; sucrose fatty acid esters, anionic, cationic, amphoteric or zwitterionic);
(b) anionic surfactants (e.g. alkylether sulfates; alkylether carboxylates; alkylbenzene sulfonates; alkylether phosphates; dialkyl sulfosuccinates; sarcosinates; alkyl sulfonates; soaps; alkyl sulfates; alkyl carboxylates; alkyl phosphates; paraffin sulfonates; secondary n-alkane sulfonates; alpha-olefin sulfonates; isethionate sulfonates; alginates);
(c) cationic surfactants (e.g. fatty amine salts; fatty diamine salts; quaternary ammonium compounds; phosphonium surfactants; sulfonium surfactants; sulfonxonium surfactants); or
(d) zwitterionic surfactants (e.g. N-alkyl derivatives of amino acids (such as glycine, betaine, aminopropionic acid); imidazoline surfactants; amine oxides; amidobetaines).

Particularly suitable surfactants for the present invention may be selected from: benzalkonium chloride, dioctyl sodium sulfosuccinate (AOT), sodium deoxycholate (NDC), polyoxyethylene-polyoxypropylene block copolymer (poloxamer), Polysorbate 20, Polysorbate 80, sorbitan monolaurate, polyethylene glycol (15)-hydroxystearate, vit-E-PEG succinate

In the present invention, the surfactant is selected from those surfactants that are capable of stabilising microparticles of glecaprevir and/or pibrentasvir in an aqueous dispersion together with a first excipient as defined herein, and which are also suitable for pharmaceutical use (e.g. they are on the US Food and Drug Administration's Center for Drug Evaluation and Research (FDA CDER) list of inactive ingredients, especially those indicated as suitable for intramuscular injection).

### Combinations of First and Second Excipient

The first and second excipient may be selected from any of those outlined above. In particular, the first and second excipient may be selected from the following combinations:
PVP and benzalkonium chloride; PVP and AOT; PVP and NDC; PVP and poloxamer; PVP and Polysorbate 20; PVP and Polysorbate 80; PVP and sorbitan monolaurate; PVP and polyethylene glycol (15)-hydroxystearate; PVP and Vit-E-PEG-succinate;
PVA and benzalkonium chloride; PVA and AOT; PVA and NDC; PVA and poloxamer; PVA and Polysorbate 20; PVA and Polysorbate 80; PVA and sorbitan monolaurate; PVA and polyethylene glycol (15)-hydroxystearate; PVA and Vit-E-PEG-succinate
HPMC and benzalkonium chloride; HPMC and AOT; HPMC and NDC; HPMC and poloxamer; HPMC and Polysorbate 20; HPMC and Polysorbate 80; HPMC and sorbitan monolaurate; HPMC and polyethylene glycol (15)-hydroxystearate; HPMC and Vit-E-PEG-succinate
PEG and benzalkonium chloride; PEG and AOT; PEG and NDC; PEG and poloxamer; PEG and Polysorbate 20; PEG and Polysorbate 80; PEG and sorbitan monolaurate; PEG and polyethylene glycol (15)-hydroxystearate; PEG and Vit-E-PEG-succinate
PVA-PEG graft copolymer and benzalkonium chloride; PVA-PEG graft copolymer and AOT; PVA-PEG graft copolymer and NDC; PVA-PEG graft copolymer and poloxamer; PVA-PEG graft copolymer and Polysorbate 20; PVA-PEG graft copolymer and Polysorbate 80; PVA-PEG graft copolymer and sorbitan monolaurate; PVA-PEG graft copolymer and polyethylene glycol (15)-hydroxystearate; PVA-PEG graft copolymer and Vit-E-PEG-succinate
poloxamer and benzalkonium chloride; poloxamer and AOT; poloxamer and NDC; are both poloxamer; poloxamer and Polysorbate 20; poloxamer and Polysorbate 80; poloxamer and sorbitan monolaurate; poloxamer and polyethylene glycol (15)-hydroxystearate; poloxamer and Vit-E-PEG-succinate
polyethoxylated castor oil and benzalkonium chloride; polyethoxylated castor oil and AOT; polyethoxylated castor oil and NDC; polyethoxylated castor oil and poloxamer; polyethoxylated castor oil and Polysorbate 20; polyethoxylated castor oil and Polysorbate 80; polyethoxylated castor oil and sorbitan monolaurate; polyethoxylated castor oil and polyethylene glycol (15)-hydroxystearate; polyethoxylated castor oil and Vit-E-PEG-succinate
sucrose and benzalkonium chloride; sucrose and AOT; sucrose and NDC; sucrose and poloxamer; sucrose and Polysorbate 20; sucrose and Polysorbate 80; sucrose and sorbitan monolaurate; sucrose and polyethylene glycol (15)-hydroxystearate; sucrose and Vit-E-PEG-succinate
lactose and benzalkonium chloride; lactose and AOT; lactose and NDC; lactose and poloxamer; lactose and Polysorbate 20; lactose and Polysorbate 80; lactose and sorbitan monolaurate; lactose and polyethylene glycol (15)-hydroxystearate; lactose and Vit-E-PEG-succinate.

The first and second excipient may be the same. It will be understood that, in embodiments wherein both the first excipient and the second excipient are the same (e.g. in the case of poloxamer, listed as both a first and a second excipient), the quantities of the first and second excipients are to be combined to arrive at the quantity of the excipient required

Combinations of first and second excipient that are particularly suitable for use in solid compositions comprising microparticles of pibrentasvir include:
polyethoxylated castor oil and benzalkonium chloride
polyethoxylated castor oil and AOT
PVP and benzalkonium chloride
PVP and AOT
PVP and sorbitan monolaurate
lactose and benzalkonium chloride
lactose and AOT
lactose and poloxamer
sucrose and benzalkonium chloride
sucrose and AOT
sucrose and sorbitan monolaurate
poloxamer and benzalkonium chloride
poloxamer and AOT
poloxamer and Polysorbate 20
PEG and benzalkonium chloride
PEG and AOT
PEG and Polysorbate 20
PEG and NDC
PVA and Polysorbate 20
PVA and Polysorbate 80
PVA and NDC
PVA and polyethylene glycol (15)-hydroxystearate
PVA and Vit-E-PEG succinate
PVA-PEG graft copolymer and AOT
PVA-PEG graft copolymer and NDC
HPMC and NDC
HPMC and AOT
HPMC and Polysorbate 20
HPMC and Polysorbate 80
HPMC and Vit-E-PEG succinate
HPMC and polyethylene glycol (15)-hydroxystearate

With preferred combinations of first and second excipient being selected from:
PVP and benzalkonium chloride
PVP and AOT
lactose and benzalkonium chloride
sucrose and AOT
poloxamer and benzalkonium chloride
poloxamer and Polysorbate 20
poloxamer and AOT
PEG and benzalkonium chloride
PVA and Polysorbate 20
PVA and Polysorbate 80
PVA and NDC
PVA and polyethylene glycol (15)-hydroxystearate
PVA and Vit-E-PEG succinate
HPMC and Polysorbate 20
HPMC and Polysorbate 80
HPMC and Vit-E-PEG succinate
HPMC and polyethylene glycol (15)-hydroxystearate
HPMC and NDC
HPMC and AOT

A particularly preferred combination of first and second excipient is PVP and AOT.

Combinations of first and second excipient that are particularly suitable for use in solid compositions comprising microparticles of glecaprevir include:
PVP and AOT
PVP and poloxamer
PVP and Polysorbate 20
PVP and Polysorbate 80
PVP and sorbitan monolaurate
lactose and AOT
sucrose and AOT
sucrose and poloxamer
sucrose and Polysorbate 20
sucrose and Polysorbate 80
poloxamer and NDC
PVA and AOT
PVA and Polysorbate 20
PVA and Polysorbate 80
PVA and NDC
PVA and polyethylene glycol (15)-hydroxystearate
PVA and Vit-E-PEG succinate
PVA-PEG graft copolymer and AOT
PVA-PEG graft copolymer and polyethylene glycol (15)-hydroxystearate
PVA-PEG graft copolymer and Polysorbate 80
HPMC and AOT
HPMC and NDC
HPMC and Polysorbate 80
HPMC and Vit-E-PEG succinate

With preferred combinations of first and second excipient being selected from:
PVP and AOT
PVP and Polysorbate 20
PVP and Polysorbate 80
PVA and AOT
PVA and Polysorbate 20
PVA and Polysorbate 80
PVA and NDC
PVA and polyethylene glycol (15)-hydroxystearate
PVA and Vit-E-PEG succinate
PVA-PEG graft copolymer and AOT
PVA-PEG graft copolymer and polyethylene glycol (15)-hydroxystearate
PVA-PEG graft copolymer and Polysorbate 80

A particularly preferred combination of first and second excipient is PVP and AOT.

Combinations of first and second excipient that are particularly suitable for use in solid compositions comprising microparticles of glecaprevir and pibrentasvir include:
PVP and AOT
PVP and sorbitan monolaurate
lactose and AOT
sucrose and AOT
poloxamer and benzalkonium chloride
poloxamer and AOT
PVA and Polysorbate 20
PVA and Polysorbate 80
PVA and NDC
PVA and polyethylene glycol (15)-hydroxystearate
PVA and Vit-E-PEG succinate
PVA-PEG graft copolymer and AOT
HPMC and AOT
HPMC and polyethylene glycol (15)-hydroxystearate
HPMC and Polysorbate 80
HPMC and Vit-E-PEG succinate

A particularly preferred combination of first and second excipient is PVP and AOT.

### Formulations of the Solid Composition

The solid composition as defined herein comprises 50 to 95 wt % of glecaprevir and/or pibrentasvir. In another embodiment, the solid composition comprises 60 to 85 wt % of glecaprevir and/or pibrentasvir. In another embodiment, the solid composition comprises 65 to 75 wt % of glecaprevir and/or pibrentasvir. Alternatively, the solid composition may comprise about 70 wt % of glecaprevir and/or pibrentasvir; preferably about 80 wt % of glecaprevir and/or pibrentasvir; more preferably about 90 wt % of glecaprevir and/or pibrentasvir. Further alternatively, the solid composition comprises 65 to 95 wt % of glecaprevir and/or pibrentasvir.

In embodiments where the solid composition comprises microparticles of glecaprevir and pibrentasvir, the mass ratio of glecaprevir to pibrentasvir may be in the range of 10:1 to 1:10, preferably in the range of 5:1 to 1:5, more preferably in the range of 3:1 to 1:3, most preferably about 1:1.

In embodiments where the solid composition comprises microparticles of glecaprevir and microparticles of pibrentasvir, the mass ratio of glecaprevir to pibrentasvir may be in the range of 10:1 to 1:10, preferably in the range of 5:1 to 1:5, more preferably in the range of 3:1 to 1:3, most preferably about 1:1.

The solid composition comprises 5 to 50 wt % of the first and second excipient combined. In another embodiment, the solid composition comprises 15 to 40 wt % of the first and second excipient combined. In another embodiment, the solid composition comprises 25 to 35 wt % of the first and second excipient combined. Alternatively, the solid composition may comprise about 30 wt % of the first and second excipient combined; preferably about 20 wt % of the first and second excipient combined; more preferably about 10 wt % of the first and second excipient combined. Further alternatively, the solid composition comprises 5 to 35 wt % of the first and second excipient combined.

The first and second excipient may be present in a mass ratio in the range of 1:1 to 4:1; preferably about 2:1 to 3:1.

The solid composition may comprise 2 to 40 wt % of the first excipient; preferably 5 to 35 wt % of the first excipient; more preferably 10 to 30 wt % first excipient. In another embodiment, the solid composition comprises about 30 wt % first excipient, preferably about 20 wt % first excipient; more preferably about 15 wt % first excipient; most preferably about 7.5 wt % first excipient. Further alternatively, the solid composition may comprise 2 to 30 wt % first excipient.

The solid composition may comprise 1 to 25 wt % second excipient; preferably 3 to 20 wt % second excipient; more preferably 5 to 18 wt % second excipient. In another embodiment, the solid composition comprises about 10 wt % second excipient; preferably about 5 wt % second excipient; more preferably about 2.5 wt % second excipient. Further alternatively, the solid composition may comprise 1 to 15 wt % second excipient.

In a particular embodiment, the solid composition comprises 40 to 90 wt % of glecaprevir and/or pibrentasvir; 2 to 40 wt % first excipient; and 1 to 25 wt % second excipient. Preferably, the solid composition comprises 50 to 80 wt % of glecaprevir and/or pibrentasvir; 10 to 40 wt % first excipient; and 5 to 20 wt % second excipient. More preferably, the solid composition comprises 60 to 70 wt % of glecaprevir and/or pibrentasvir; 15 to 35 wt % first excipient; and 5 to 15 wt % second excipient.

Alternatively, the solid composition comprises 40 to 80 wt % glecaprevir and/or pibrentasvir; 5 to 50 wt % of the first excipient; and 1 to 30 wt % of the second excipient.

Further alternatively, the solid composition comprises 50 to 70 wt % glecaprevir and/or pibrentasvir; 20 to 40 wt % of the first excipient; and 5 to 15 wt % of the second excipient. Yet further alternatively the solid composition comprises about 60 wt % glecaprevir and/or pibrentasvir; about 30 wt % of the first excipient; and about 10 wt % of the second excipient.

Unless otherwise stated, the above weight percentages relate to the % by weight of a particular constituent as a proportion of the total weight of the solid composition.

The solid composition may comprise additional excipients, for instance, to further facilitate dispersion or stabilisation of dispersions of the microparticles in an aqueous medium, a pharmaceutically acceptable diluent or in vivo.

### Process for Preparing the Solid Compositions

Solid compositions of the present invention may be prepared by a number of methods well known in the art, including ‘top-down’ physical methods such as nano (or bead) milling and high pressure homogenisation. Other suitable techniques for forming such compositions are described in general terms in Horn and Reiger, Angew. Chem. Int. Ed., 2001, 40, 4330-4361.

It is generally preferred that the solid compositions of the present invention are prepared by an oil-in-water emulsion technique whereby the glecaprevir and/or pibrentasvir are dissolved in the oil phase and the first and second excipients are present in the water phase. The oil and water solvents are then removed by freeze drying, spray drying or spray granulation to provide a solid composition according to the invention.

As previously highlighted, in some embodiments where the solid composition comprises both glecaprevir and pibrentasvir, individual microparticles may comprise both glecaprevir and pibrentasvir, in which case both glecaprevir and pibrentasvir are present during nanoparticle formation. In alternative embodiments where the solid composition comprises both glecaprevir and pibrentasvir, the solid composition may comprise individual microparticles of glecaprevir and individual microparticles of pibrentasvir, in which case each set of individual microparticles are formed separately before being subsequently mixed or blended. A mixture of these two processes is also feasible.

Thus, in accordance with one aspect of the present invention, there is provided a general process for preparing a solid composition comprising microparticles of glecaprevir and pibrentasvir as defined herein, the process comprising:
(a) preparing an oil-in-water emulsion comprising:
an oil phase comprising both glecaprevir and pibrentasvir; and
an aqueous phase comprising a first excipient and a second excipient, each as defined herein; and
(b) removing the oil and water from the oil-in-water emulsion to form the solid composition.

In accordance with one embodiment, there is provided a process for preparing a solid composition comprising microparticles of glecaprevir as defined herein, the process comprising:
(a) preparing a glecaprevir oil-in-water emulsion comprising:
an oil phase comprising glecaprevir; and
an aqueous phase comprising a first excipient and a second excipient, each as defined herein; and
(b) removing the oil and water from the glecaprevir oil-in-water emulsion to form the solid composition.

In accordance with one embodiment, there is provided a process for preparing a solid composition comprising microparticles of pibrentasvir as defined herein, the process comprising:
(a) preparing a pibrentasvir oil-in-water emulsion comprising:
an oil phase comprising pibrentasvir; and
an aqueous phase comprising a first excipient and a second excipient, each as defined herein; and
(b) removing the oil and water from the pibrentasvir oil-in-water emulsion to form the solid composition.

In accordance with one embodiment, there is provided a process for preparing a solid composition comprising microparticles of glecaprevir and microparticles of pibrentasvir as defined herein, the process comprising:
(a) preparing a glecaprevir oil-in-water emulsion comprising:
an oil phase comprising glecaprevir; and
an aqueous phase comprising a first excipient and a second excipient, each as defined herein;
(b) preparing a pibrentasvir oil-in-water emulsion comprising:
an oil phase comprising pibrentasvir; and
an aqueous phase comprising a first excipient and a second excipient, each as defined herein;
(c) removing the oil and water from the glecaprevir oil-in-water emulsion to form a glecaprevir solid product;
(d) removing the oil and water from the pibrentasvir oil-in-water emulsion to produce a pibrentasvir solid product;
(e) blending together the glecaprevir and pibrentasvir solid products to form the solid composition.

In accordance with one embodiment, there is provided a process for preparing a solid composition comprising microparticles of glecaprevir and microparticles of pibrentasvir as defined herein, the process comprising:
(a) preparing a glecaprevir oil-in-water emulsion comprising:
an oil phase comprising glecaprevir; and
an aqueous phase comprising a first excipient and a second excipient, each as defined herein;
(b) preparing a pibrentasvir oil-in-water emulsion comprising:
an oil phase comprising pibrentasvir; and
an aqueous phase comprising a first excipient and a second excipient, each as defined herein;
(c) combining the glecaprevir oil-in-water emulsion with the pibrentasvir oil-in-water emulsion to form a mixed oil-in-water emulsion;
(d) removing the oil and water from the mixed oil-in-water emulsion to form the solid composition.

An advantage of the processes of the present invention is that the emulsions formed in the initial steps are sufficiently homogenous and stable to allow for effective and uniform drying upon removal of the oil and water. Furthermore, the microparticles formed are substantially uniform in their physical form (size, shape etc.).

The oil-in-water formation steps may be performed by methods well-known in the art. Any suitable method for forming the oil-in-water emulsions may therefore be used. In particular, mixing of the oil and water phases to form the oil-in-water emulsion may be performed by methods well known in the art. For example, the mixing may involve stirring, sonication, homogenisation, or a combination thereof. In a particular embodiment, the mixing is facilitated by sonication and/or homogenisation.

The oil-in-water formation steps may be performed, for example, by using the methods described in WO 2004/011537 A1 (COOPER et al), which is hereby duly incorporated by reference.

In a particular embodiment, oil-in-water emulsion formation comprises:
(i) providing an oil phase comprising glecaprevir and/or pibrentasvir;
(ii) providing an aqueous phase comprising the first excipient and second excipient; and
(iii) mixing the oil phase and aqueous phase to produce the oil-in-water emulsion.

Suitably, the oil phase is provided by dissolving glecaprevir and/or pibrentasvir in a suitable organic solvent. In embodiments where glecaprevir and pibrentasvir are dissolved, each of glecaprevir and pibrentasvir may be dissolved separately in a suitable organic solvent (optionally different organic solvents) and subsequently mixed to form the oil phase. Alternatively, they may be dissolved simultaneously.

Suitably, the aqueous phase is provided by dissolving first excipient and second excipient in an aqueous medium, preferably in water. Alternatively the aqueous phase may be provided by mixing two separately prepared aqueous solutions of the first excipient and second excipient.

In a particular embodiment, further aqueous medium (e.g. water) or organic solvent is added before or during mixing step (iii).

The concentration of glecaprevir and/or pibrentasvir in the oil-in-water emulsion is suitably as concentrated as possible to facilitate effective scale-up of the process. For example, the concentration of glecaprevir and/or pibrentasvir in the oil phase is suitably 50 mg/ml or higher, more suitably 100 mg/ml or higher, even more suitably 150 mg/ml or higher, most suitably greater than 170 mg/ml.

The concentration of the first excipient in the aqueous phase is suitably 0.1-50 mg/mL, more suitably 0.5 to 25 mg/mL, even more suitably 1 to 10 mg/mL.

The concentration of the second excipient in the aqueous phase is suitably 0.1 to 25 mg/mL, more suitably 0.2 to 10 mg/mL, even more suitably 0.5 to 5 mg/mL.

The mass ratio of the first excipient to second excipient in the aqueous phase may be in the range of 9:1 to 1:6; preferably in the range of 7:1 to 4:1.

The organic solvent forming the oil phase is (substantially) immiscible with water. Suitably the organic solvent is aprotic. Suitably the organic solvent has a boiling point less than 120° C., suitably less than 100° C., suitably less than 90° C.

In a particular embodiment, the organic solvent is a selected from the Class 2 or 3 solvents listed in the International Conference on Harmonization (ICH) guidelines relating to residual solvents.

In a particular embodiment, the organic solvent is selected from chloroform, dichloromethane, dichloroethane, tetrachloroethane, cyclohexane, hexane(s), isooctane, dodecane, decane, methylbutyl ketone (MBK), methylcyclohexane, tetrahydrofuran, toluene, xylene, butyl acetate, mineral oil, tert-butylmethyl ether, heptanes(s), isobutyl acetate, isopropyl acetate, methyl acetate, methylethyl ketone, ethyl acetate, ethyl ether, pentane, and propyl acetate, or any suitably combination thereof.

In a particular embodiment, the organic solvent is selected from dichloromethane and ethyl acetate.

The organic solvent may include a cosolvent. The cosolvent may be selected from C1 to C4 alcohols (such as methanol, ethanol, n-propanol, iso-propanol, n-butanol, sec-butanol, and iso-butanol) and C5 to C7 alkanes (such as pentane, hexane, and heptane).

Preferred combinations of organic solvent and cosolvent include DCM and methanol, DCM and ethanol, DCM and isopropanol, and DCM and hexane.

The oil phase may comprise between 1 and 30 v/v % of the oil-in-water emulsion. Preferably, the oil phase comprises between 5 and 20 v/v % of the oil-in-water emulsion, most preferably between 5 and 10 v/v % of the oil-in-water emulsion. In an embodiment, the oil phase comprises 7 v/v % of the oil-in-water emulsion.

Mixing step (iii) suitably produces a substantially uniform oil-in-water emulsion. As previously indicated, mixing may be performed using methods well known in the art. Suitably, mixing step (iii) involves stirring, sonication, homogenisation, or a combination thereof. In a particular embodiment, mixing step (iii) involves sonication and/or homogenisation.

Removing the oil and water may be performed using methods well known in the art. Suitably removing the oil and water involves freeze drying, spray drying or spray granulation.

Removing the oil and water may be performed using methods described in WO 2004/011537 A1 (COOPER et al), the entire contents of which are hereby incorporated by reference.

In a particular embodiment, removing the oil and water involves freeze drying the oil-in-water emulsion. As such, removing the oil and water may suitably comprise freezing the oil-in-water emulsion and then removing the solvents under vacuum.

Preferably, the freezing of the oil-in-water emulsion may be performed by externally cooling the oil-in-water emulsion. For example, a vessel containing the oil-in-water emulsion may be externally cooled, for example, by submerging the vessel in a cooling medium, such as liquid nitrogen. Alternatively, the vessel containing the oil-in-water emulsion may be provided with an external “jacket” through which coolant is circulated to freeze the oil-in-water emulsion. Alternatively, the vessel may comprise an internal element through which coolant is circulated in order to freeze the oil-in-water emulsion.

In a further alternative, the oil-in-water emulsion is frozen by being contacted directly with a cooling medium at a temperature effective for freezing the emulsion. In such cases, the cooling medium (e.g. liquid nitrogen) may be added to the oil-in-water emulsion, or the oil-in-water emulsion may be added to the cooling medium. In a particular embodiment, the oil-in-water emulsion is added to the fluid medium (e.g. liquid nitrogen), suitably in a dropwise manner. This order of addition provides higher purities of final product. As such, frozen droplets of the oil-in-water emulsion may suitably form. Such frozen droplets may suitably be isolated (e.g. under vacuum to remove the fluid medium/liquid nitrogen). The solvent is then suitably removed from the frozen droplets under vacuum. The resulting solid composition is then isolated.

It is preferred that the oil and water are removed by spray drying as this removes the need to freeze the oil-in-water emulsion, with the microparticulate nature of the solid composition being retained through the rapid nature of the solvent evaporation.

In processes where solid compositions comprising microparticles of glecaprevir and solid compositions comprising microparticles of pibrentasvir require blending to produce the solid composition comprising microparticles of glecaprevir and microparticles of pibrentasvir, the blending may suitably employ methods well known in the art. Blending suitably provides a substantially homogeneous solid composition.

The present invention also provides a solid composition obtainable by, obtained by, or directly obtained by any of the processes described herein.

### Aqueous Dispersion of Microparticles of Glecaprevir and/or Pibrentasvir

The present invention also provides an aqueous dispersion, obtainable by, obtained by, or directly obtained by dispersing the solid composition as defined herein in an aqueous medium. When the solid composition is dispersed in the aqueous medium, the first and second excipients are dissolved within the aqueous medium to release the microparticles of glecaprevir and/or pibrentasvir in a dispersed form. The microparticles of glecaprevir and/or pibrentasvir, which were formerly dispersed within a solid mixture of the first and second excipients, then become dispersed within the aqueous medium and are stabilized by the first and second excipients, thereby preventing premature coagulation and aggregation.

In a particular embodiment, the aqueous medium comprises 20 to 99.5 wt % of the total aqueous dispersion. In a particular embodiment, the aqueous medium comprises 50 to 98 wt % of the total aqueous dispersion. In a particular embodiment, the aqueous medium comprises 70 to 95 wt % of the total aqueous dispersion. Suitably, the remaining proportion of the aqueous dispersion consists essentially of glecaprevir and/or pibrentasvir, first excipient, and second excipient, whose proportions within the aqueous dispersion as a whole are accordingly calculated (and scaled) by reference to the proportions recited in relation to the solid composition.

For aqueous dispersions of microparticles of glecaprevir, the concentration of glecaprevir may be at least 150 mg/mL, preferably at least 200 mg/mL, more preferably at least 300 mg/mL, and most preferably at least 500 mg/mL. Alternatively, the concentration of glecaprevir may be 100 to 1000 mg/mL, preferably 200 to 900 mg/mL, more preferably 300 to 800 mg/mL, and most preferably 500 to 700 mg/mL.

For aqueous dispersions of microparticles of pibrentasvir, the concentration of pibrentasvir may be at least 150 mg/mL, preferably at least 200 mg/mL, more preferably at least 300 mg/mL, and most preferably at least 500 mg/mL. Alternatively, the concentration of pibrentasvir may be 100 to 1000 mg/mL, preferably 200 to 900 mg/mL, more preferably 300 to 800 mg/mL, and most preferably 500 to 700 mg/mL.

For aqueous dispersions of microparticles of glecaprevir and pibrentasvir, the combined concentration of glecaprevir and pibrentasvir may be at least 150 mg/mL, preferably at least 200 mg/mL, more preferably at least 300 mg/mL, and most preferably at least 500 mg/mL. Alternatively, the combined concentration of glecaprevir and pibrentasvir may be 100 to 1000 mg/mL, preferably 200 to 900 mg/mL, more preferably 300 to 800 mg/mL, and most preferably 500 to 700 mg/mL. In such embodiments, the mass ratio of glecaprevir to pibrentasvir may be in the range of 10:1to 1:2, preferably in the range of 5:1 to 1:1, more preferably about 2.5:1.

In a particular embodiment, the aqueous medium is water. In an alternative embodiment, the aqueous medium comprises water and one or more additional pharmaceutically acceptable diluents or excipients. In particular embodiments, the aqueous medium comprises saline or a phosphate buffered saline (PBS).

The microparticles of the present invention have an average particle diameter of less than 5 micron (μm). In a particular embodiment, the microparticles have an average particle diameter of between 10 nm and 2500 nm, preferably between 20 nm and 2000 nm, more preferably between 50 nm and 1500 nm, further preferably between 100 nm and 1000 nm, and most preferably between 100 and 500 nm. In embodiments, the microparticles are nanoparticles (i.e. they have a particle diameter in the range of 1 to 1000 nm).

The microparticles of the present invention may have a polydispersity less than or equal to 0.8, preferably less than or equal to 0.6, more preferably less than or equal to 0.5.

The particle diameter and polydispersity of the microparticles may be assessed by any suitable technique known in the art (e.g. laser diffraction, laser scattering, electron microscopy). In an embodiment of the invention, particle diameter is assessed by dispersing the solid composition in an aqueous medium and determining the particle diameter with a Malvern Panalytical Limited Zetasizer Ultra.

The aqueous dispersion may be used in the formulation of a microneedle array.

### Process for Preparing an Aqueous Dispersion

The present invention provides a process for preparing an aqueous dispersion, the process comprising dispersing a solid composition as defined herein in an aqueous medium.

In a particular embodiment, the aqueous medium is water. In an alternative embodiment, the aqueous medium comprises water and one or more additional excipients. In particular embodiments, the aqueous medium comprises saline or a phosphate buffered saline (PBS).

Dispersing the solid composition in the aqueous medium may comprise adding the solid composition to an aqueous medium and suitably agitating the resulting mixture (e.g. by shaking, homogenisation, sonication, stirring, etc.).

### Pharmaceutical Compositions

The present invention provides a pharmaceutical composition comprising a solid composition or an aqueous dispersion as defined herein. The pharmaceutical compositions of the present invention may further comprise one or more additional pharmaceutically acceptable excipients.

The solid compositions of the invention may be formulated into a form suitable for oral use (for example as tablets, lozenges, hard or soft capsules, or dispersible powders or granules) by techniques known in the art. As such, the solid compositions of the invention may be mixed with one or more additional pharmaceutical excipients during this process, such as antiadherants, binders, coatings, enterics, disintegrants, fillers, diluents, flavours, colours, lubricants, glidants, preservatives, sorbents, and sweeteners.

In a particular embodiment, the pharmaceutical composition is a tablet or capsule comprising the solid composition.

The aqueous dispersion of the present invention may be administered as it is or further formulated with one or more additional excipients to provide a dispersion, elixir or syrup that is suitable for oral use, or a dispersion that is suitable for parenteral administration (for example, a sterile aqueous dispersion for intravenous, subcutaneous, intramuscular, intraperitoneal or intramuscular dosing).

In a particular embodiment, the pharmaceutical composition is an aqueous dispersion as described herein. Such dispersed formulations can be used to accurately measure smaller dosages, such as those suitable for administration to children.

In a particular embodiment, the pharmaceutical composition is in a form suitable for parenteral delivery, whether via subcutaneous or intramuscular delivery.

It will be appreciated that different pharmaceutical compositions of the invention may be obtained by conventional procedures, using conventional pharmaceutical excipients, well known in the art.

The pharmaceutical compositions of the invention contain a therapeutically effective amount of glecaprevir and/or pibrentasvir. A person skilled in the art will know how to determine and select an appropriate therapeutically effective amount of glecaprevir and/or pibrentasvir to include in the pharmaceutical compositions of the invention.

### Injectable Formulations

The present invention provides an injectable formulation comprising the solid composition as described herein, the aqueous dispersion as described herein, or the pharmaceutical composition as described herein. In embodiments, the injectable formulation is intramuscularly injectable. In other embodiments, the injectable formulation is subcutaneously injectable.

Said formulations may be in solid form (or substantially solid form, e.g. a paste) or liquid form or semi-solid form, in which the glecaprevir and/or pibrentasvir is present in the form of microparticles. The microparticles of glecaprevir and/or pibrentasvir may be dispersed within one or more carrier materials. When in liquid form, each microparticle of glecaprevir and/or pibrentasvir is stabilised by the first and second excipients.

### Long Acting Injectable Formulations

The present invention provides a long acting injectable formulation comprising the aqueous dispersion as described herein, or the pharmaceutical composition as described herein. The long acting injectable formulation may be intramuscularly injectable or it may be subcutaneously injectable. The long acting injectable formulation may, in addition to the microparticles of glecaprevir and/or pibrentasvir and the aqueous medium, further comprise one or more additional pharmaceutically acceptable excipients, such as thickeners, preservatives, and stabilizers.

The injectable formulations of microparticles of glecaprevir and/or pibrentasvir may be long acting injectable formulations. Such formulations are advantageously designed for administration as an intramuscular or a subcutaneous injection that forms a depot at the site of the injection. Long acting injectable formulations improve adherence to prophylaxis and/or treatment, especially in respect of viral illnesses which require extended treatment durations, such as HCV infection, and the consequences that ensue. Furthermore, a depot injection is beneficial in that it may be easier to administer than conventional preparations and allows for simpler follow-up/on-going care.

The long acting injectable formulations of the present invention permit the formation of a depot of microparticles of glecaprevir and/or pibrentasvir as these drugs are poorly soluble in aqueous media, preventing them from rapidly exiting the depot and entering the bloodstream of the patient. Generally, long acting injectable formulations are administered intramuscularly or subcutaneously to certain sites, such as the deltoid, dorsogluteal, ventrogluteal, vastus lateralis, and rectus femoris muscles. Each site has a limit on the maximum volume that may be injected without prompting excessive discomfort in the patient or losing effectiveness. The maximum volume, even for the larger sites, is generally only as high as 3 mL. The administration also takes longer than a standard injection, typically taking 10 seconds to administer 1 mL.

This limitation on the number of suitable sites and volumes permitted in each means that it is important for the long acting injectable formulations to contain high concentrations of glecaprevir and/or pibrentasvir, so as to minimise the volume (and hence number and duration of injections) required to provide a depot capable of releasing a therapeutically effective amount of glecaprevir and/or pibrentasvir.

In addition, without wishing to be bound by theory, the glecaprevir and/or pibrentasvir are thought to be released from the depot at a rate defined by their physicochemical properties (e.g. solubility controlling the rate at which each of the glecaprevir and/or pibrentasvir dissolve from the surface of the microparticles) and the local physiological environment. There is, therefore, a minimum concentration of glecaprevir and/or pibrentasvir that must be present in the depot in order to ensure that, as the glecaprevir and/or pibrentasvir is released at the rate defined by their physicochemical properties and local physiological environments (which may themselves be further influenced by the introduction of the depot, e.g. through formulation of a granuloma around the depot), the amount that is released is a therapeutically effective amount (i.e. the concentration of glecaprevir and/or pibrentasvir in the formulation must be sufficient to produce a therapeutically effective concentration of glecaprevir and/or pibrentasvir in vivo). Furthermore, the therapeutically effective amount of glecaprevir and/or pibrentasvir must be maintained for the duration of the treatment, meaning that the depot must contain all of the glecaprevir and/or pibrentasvir required for the duration of the treatment.

For long acting injectable formulations of microparticles of glecaprevir, the concentration of glecaprevir may be at least 150 mg/mL, preferably at least 200 mg/mL, more preferably at least 300 mg/mL, and most preferably at least 500 mg/mL. Alternatively, the concentration of glecaprevir may be 100 to 1000 mg/mL, preferably 200 to 900 mg/mL, more preferably 300 to 800 mg/mL, and most preferably 500 to 700 mg/mL.

For long acting injectable formulations of microparticles of pibrentasvir, the concentration of pibrentasvir may be at least 150 mg/mL, preferably at least 200 mg/mL, more preferably at least 300 mg/mL, and most preferably at least 500 mg/mL. Alternatively, the concentration of pibrentasvir may be 100 to 1000 mg/mL, preferably 200 to 900 mg/mL, more preferably 300 to 800 mg/mL, and most preferably 500 to 700 mg/mL.

For long acting injectable formulations of microparticles of glecaprevir and pibrentasvir, the combined concentration of glecaprevir and pibrentasvir may be at least 150 mg/mL, preferably at least 200 mg/mL, more preferably at least 300 mg/ml, and most preferably at least 500 mg/mL. Alternatively, the combined concentration of glecaprevir and pibrentasvir may be 100 to 1000 mg/mL, preferably 200 to 900 mg/mL, more preferably 300 to 800 mg/mL, and most preferably 500 to 700 mg/mL.

Preferably, glecaprevir and/or pibrentasvir are released into the bloodstream of the patient from the depot of microparticles of glecaprevir and/or pibrentasvir at a controlled rate such that a therapeutically effective amount of glecaprevir and/or pibrentasvir is achieved over a period of at least about two weeks from the date of administration. Further preferably the therapeutically effective amount of glecaprevir and/or pibrentasvir is achieved for at least about three weeks, more preferably at least about one month, most preferably at least about two months from the date of administration of the injection.

### Implantable Rods

The present invention provides implantable rods comprising microparticles of glecaprevir and/or pibrentasvir dispersed in a monolith of the first excipient and the second excipient.

The microparticles of glecaprevir and/or pibrentasvir may be microparticles of glecaprevir in the absence of pibrentasvir. Alternatively, the microparticles of glecaprevir and/or pibrentasvir may be microparticles of pibrentasvir in the absence of glecaprevir.

The microparticles of glecaprevir and/or pibrentasvir may be microparticles of glecaprevir and/or microparticles of pibrentasvir. In other words, the solid composition comprises microparticles consisting of, or consisting essentially of, glecaprevir and/or microparticles consisting of, or consisting essentially of, pibrentasvir.

Alternatively, microparticles of glecaprevir and/or pibrentasvir may be microparticles of glecaprevir and pibrentasvir, wherein the microparticles comprise both glecaprevir and pibrentasvir.

In certain embodiments, the implantable rods may comprise a mixture of the above microparticles. In other words, the implantable rods may comprise at least two of microparticles of glecaprevir, microparticles of pibrentasvir, and microparticles of both glecaprevir and pibrentasvir.

The glecaprevir and/or pibrentasvir which comprises the microparticles may be amorphous (i.e. substantially non-crystalline in nature).

The microparticles of the present invention have an average particle diameter of less than 5 micron (μm). In a particular embodiment, the microparticles have an average particle diameter of between 10 nm and 2500 nm, preferably between 20 nm and 2000 nm, more preferably between 50 nm and 1500 nm, further preferably between 100 nm and 1000 nm, and most preferably between 100 and 500 nm. In embodiments, the microparticles are nanoparticles (i.e. they have a particle diameter in the range of 1 to 1000 nm). It will be understood that references to particle diameter are references to the Z-average hydrodynamic diameter of the microparticles.

The microparticles of the present invention may have a polydispersity less than or equal to 0.8, preferably less than or equal to 0.6, more preferably less than or equal to 0.5.

The particle diameter and polydispersity of the microparticles may be assessed by any suitable technique known in the art (e.g. laser diffraction, laser scattering, electron microscopy). In an embodiment of the invention, particle diameter (i.e. Z-average hydrodynamic diameter) is assessed by dispersing the solid composition in an aqueous medium and determining the particle diameter with a Malvern Panalytical Limited Zetasizer Ultra.

The monolith of the first excipient and second excipient is non-porous in nature.

The first excipient may be as described for the solid composition of the present invention. In embodiments, the first excipient is selected from polyvinylpyrrolidone (PVP), polyvinyl alcohol (PVA), polyethylene glycol (PEG), hydroxypropyl methylcellulose (HPMC), polyoxyethylene-polyoxypropylene block copolymer (poloxamer), polyethoxylated castor oil, PVA-PEG graft copolymer, lactose, and sucrose.

The second excipient may be as described for the solid composition of the present invention. In embodiments, the second excipient is selected from dioctyl sodium sulfosuccinate (AOT), benzalkonium chloride, sodium deoxycholate (NDC), poloxamer, Polysorbate 20, Polysorbate 80, sorbitan monolaurate, polyethylene glycol (15)-hydroxystearate, and vit-E-PEG succinate.

The first and second excipients may be used in any combination as described for the solid composition of the present invention.

The relative quantities of glecaprevir and/or pibrentasvir, first excipient, and second excipient may be as described herein for the solid composition of the present invention.

The implantable rod is suitable for implantation into a patient (e.g. subcutaneously). The rods may be of any suitable shape or dimension for implantation. In embodiments, the rods are cylindrical. The length of the rods may be between 1 and 100 mm, preferably between 1 and 80 mm, more preferably between 2 and 50 mm, yet more preferably between 5 and 40 mm, and most preferably between 10 and 20 mm. The diameter of the rods may be between 0.1 and 5 mm, preferably between 0.5 and 2.5 mm, more preferably between 1 and 2 mm.

### Processes for Preparing Implantable Rods

Implantable rods may be prepared by any suitable process for the conversion of fine thermoplastic solids to cohered monoliths, such as injection moulding, extruding and other such methods known to those skilled in the art. One suitable method comprises compressing the solid compositions of the first aspect of the present invention while heating in order to collapse the porous matrix of first and second excipient and to cohere discrete particles to form the monolith. Removing the porosity increases the density of the composition, making it easier to handle and making it viable to insert the composition as an implant. This also increases the time taken to dissolve the composition. Without wishing to be bound by theory, it is the experience of the inventors that the microparticulate nature of active compounds, such as glecaprevir and pibrentasvir, is retained in the implantable rods following conversion. Using the solid compositions of the present invention to produce the implants makes higher concentrations of glecaprevir and/or pibrentasvir accessible, while also retaining the highly dispersible nature of the microparticle formulation.

The compressive force may be applied under a reduced pressure atmosphere (e.g. a vacuum). Doing so assists in the removal of any remaining volatile substances, such as solvents, and reduces the incidence of bubbles by removing any gas that is entrained in the solid composition. Certain apparatus may also use the pressure differential to apply the compressive force to the solid composition.

Heating the compressed solid composition requires increasing the temperature of the solid composition such that discrete volumes of the first and second excipients cohere under the pressure of the compression step. However, the temperature must be below that which would deteriorate the glecaprevir and/or pibrentasvir. The compressed solid composition may be heated to a temperature from 60 to 160° C., preferably from 80 to 140° C., more preferably from 100 to 120° C., most preferably about 110° C. The elevated temperature is maintained for a period of from 1 minute to 30 minutes, preferably from 2 minutes to 25 minutes, more preferably from 5 minutes to 15 minutes, most preferably about 10 minutes. The compressive force and/or vacuum is preferably maintained during the heating step. Any suitable means may be used to supply heat for the heating step. For example, an electrical heater, such as a hotplate.

Following the heating step, the rod may, optionally, be cooled. For example, through contact with a cold surface.

### Microneedle Arrays

The present invention provides microneedle arrays comprising microneedles of a first composition arrayed on one face of a baseplate of a second composition, wherein the first composition comprises microparticles of glecaprevir and/or pibrentasvir dispersed within a monolith comprising a first excipient, a second excipient and at least one structural polymer. By using microparticles of glecaprevir and/or pibrentasvir, especially those present in the solid composition of the present invention, higher loadings of the water-insoluble drugs may be obtained, while also retaining their highly dispersible nature.

The microparticles of glecaprevir and/or pibrentasvir may be microparticles of glecaprevir in the absence of pibrentasvir. Alternatively, the microparticles of glecaprevir and/or pibrentasvir may be microparticles of pibrentasvir in the absence of glecaprevir.

The microparticles of glecaprevir and/or pibrentasvir may be microparticles of glecaprevir and/or microparticles of pibrentasvir. In other words, the solid composition comprises microparticles consisting of, or consisting essentially of, glecaprevir and/or microparticles consisting of, or consisting essentially of, pibrentasvir.

Alternatively, microparticles of glecaprevir and/or pibrentasvir may be microparticles of glecaprevir and pibrentasvir, wherein the microparticles comprise both glecaprevir and pibrentasvir.

In certain embodiments, the solid composition may comprise a mixture of the above microparticles. In other words, the solid composition may comprise at least two of microparticles of glecaprevir, microparticles of pibrentasvir, and microparticles of both glecaprevir and pibrentasvir.

The glecaprevir and/or pibrentasvir which comprises the microparticles may be amorphous (i.e. substantially non-crystalline in nature).

The microparticles of the present invention have an average particle diameter of less than 5 micron (μm). In a particular embodiment, the microparticles have an average particle diameter of between 10 nm and 2500 nm, preferably between 20 nm and 2000 nm, more preferably between 50 nm and 1500 nm, further preferably between 100 nm and 1000 nm, and most preferably between 100 and 500 nm. In embodiments, the microparticles are nanoparticles (i.e. they have a particle diameter in the range of 1 to 1000 nm). It will be understood that references to particle diameter are references to the Z-average hydrodynamic diameter of the microparticles.

The microparticles of the present invention may have a polydispersity less than or equal to 0.8, preferably less than or equal to 0.6, more preferably less than or equal to 0.5.

The particle diameter and polydispersity of the microparticles may be assessed by any suitable technique known in the art (e.g. laser diffraction, laser scattering, electron microscopy). In an embodiment of the invention, particle diameter (i.e. Z-average hydrodynamic diameter) is assessed by dispersing the solid composition in an aqueous medium and determining the particle diameter with a Malvern Panalytical Limited Zetasizer Ultra.

The monolith of the first excipient and second excipient is non-porous in nature.

The first excipient may be as described for the solid composition of the present invention. In embodiments, the first excipient is selected from polyvinylpyrrolidone (PVP), polyvinyl alcohol (PVA), polyethylene glycol (PEG), hydroxypropyl methylcellulose (HPMC), polyoxyethylene-polyoxypropylene block copolymer (poloxamer), polyethoxylated castor oil, PVA-PEG graft copolymer, lactose, and sucrose.

The second excipient may be as described for the solid composition of the present invention. In embodiments, the second excipient is selected from dioctyl sodium sulfosuccinate (AOT), benzalkonium chloride, sodium deoxycholate (NDC), poloxamer, Polysorbate 20, Polysorbate 80, sorbitan monolaurate, polyethylene glycol (15)-hydroxystearate, and vit-E-PEG succinate.

In principle, any hydrophilic polymer suitable for use in pharmaceutical formulations may be employed as a structural polymer, for example, the polymers that are suitable as first excipients. In embodiments, the structural polymer is selected to be the same as a first excipient, as this helps to ensure compatibility. In embodiments, the structural polymer is selected from PVA, PVP, and combinations thereof. Polymers with MW below 60 kDa (for example, PVA with MW of 9-10 and PVP with MW of 58 kDa) are preferred as they are known to be swiftly eliminated from the human body via renal excretion. The purpose of the structural polymer is to provide the microneedles with sufficient mechanical strength to enable insertion into skin and consequent delivery of the glecaprevir and/or pibrentasvir.

The relative quantities of glecaprevir and/or pibrentasvir, first excipient, and second excipient may be as described herein for the solid composition of the present invention. The ratio of the glecaprevir and/or pibrentasvir, first excipient, and second excipient to the structural polymer may be in the range of 10:1 to 10:5, preferably in the range of 10:2 to 10:4, most preferably the ratio is about 10:3, such as 10:3.2. Overall, the first composition may comprise 4 to 8 parts glecaprevir and/or pibrentasvir, 2 to 4 parts first excipient, 0.5 to 2 parts second excipient, and 2 to 5 parts structural polymer. In one embodiment, the first composition comprises 6 parts glecaprevir and/or pibrentasvir, 3 parts first excipient, 1 part second excipient, and 3.2 parts structural polymer.

The baseplate comprises a base polymer. In principle, any hydrophilic polymer suitable for use in pharmaceutical formulations may be employed as a base polymer, for example, the polymers that are suitable as first excipients. In embodiments, the base polymer is selected to be the same as a first excipient and/or structural polymer. In embodiments, the base polymer is PVP. Typically, the base polymer will comprise high molecular weight polymer, such as PVP with a MW of 360 kDa, to impart a degree of rigidity to the base plate. The base plate may further comprise one or more additives to improve the properties of the base plate, for example, a low molecular weight polyol, such as glycerol, may reduce brittleness of the base plate. If present, the additive and base polymer are used in a ratio from 1:40 to 1:10, preferably a ratio of 1:30 to 1:15, more preferably a ratio of about 1:20.

The relative quantities of first composition and second composition is determined by the physical dimensions of the microneedles relative to the base plate, with the majority of the microneedle volume comprising first composition and the remaining microneedle volume and base plate volume comprising second composition. In embodiments, at least 50% of the microneedle volume comprises first composition, preferably at least 60%, further preferably at least 80%, more preferably at least 90%, most preferably substantially all of the microneedle volume comprises first composition.

By control of the loading of glecaprevir and/or pibrentasvir and the dimensions of the microneedle array, the microneedle array may be designed to contain substantially any suitable amount of glecaprevir and/or pibrentasvir. In embodiments, the microneedle array contains between 1 and 20 mg of glecaprevir and/or pibrentasvir, preferably between 2 and 10 mg, more preferably about 5 mg.

The dimensions of the microneedles and microneedle array are determined by the mould used in their production and can have substantially and suitable dimension. The heights of the microneedles may be in the range of 50 to 1000 μm, preferably in the range of 500 to 900 μm. The base width of the microneedles may be in the range of 50 to 500 μm, preferably in the range of 100 to 300 μm. The interspacing between the needles may be in the range of 50 to 200 μm, preferably about 100 μm. The area microneedle array may be in the range of 0.1 to 100 cm², preferably in the range of 0.5 to 30 cm². The microneedle array may comprise between 2 and 2000 microneedles. Typically, the microneedles are arrayed in a grid, but substantially any arrangement may be used.

### Processes for Preparing Implantable Rods

Microneedle arrays may be prepared by any suitable process known to those skilled in the art. One suitable method comprises the steps of: a) dispersing a solid composition according to the present invention and at least one structural polymer in a solvent to form a microneedle precursor dispersion; b) placing the microneedle precursor dispersion into a mould; c) compressing the microneedle precursor dispersion in the mould and then drying to form microneedles; d) adding a baseplate precursor solution into the mould; e) compressing the baseplate precursor solution and then drying to form the baseplate; and f) releasing the microneedle array from the mould. Without wishing to be bound by theory, it is the experience of the inventors that the microparticulate nature of active compounds, such as glecaprevir and pibrentasvir, is retained in the microneedles. Using the solid compositions of the present invention to produce the microneedle arrays allows for higher loading of the water insoluble drugs, while allowing them to remain in their water dispersible microparticulate form.

The step of dispersing the solid composition according to the present invention and at least one structural polymer may comprise individual steps of dispersing the solid composition in a first quantity of the solvent, dissolving the at least one structural polymer in a second quantity of the solvent, and then mixing. The microneedle precursor dispersion may comprise the solid composition in an amount of between 10and 50 wt %, preferably between 20 and 40 wt %, more preferably between 25 and 35 wt %, most preferably about 30 wt %. The microneedle precursor dispersion may comprise the at least one structural polymer in an amount between 1 and 20 wt %, preferably between 5 and 15 wt %, more preferably about 10 wt %.

The mould contains microcavities that correspond to the shape of the microneedles. The steps of placing the microneedle precursor dispersion into the mould, compressing and drying to form the microneedles may not fill the cavities of the mould. Accordingly, these steps may be repeated so as to increase the volume of the cavities that are filled.

The baseplate precursor solution comprises a base polymer, a solvent, and, optionally, one or more additives. The solution may comprise between 10 and 50 wt % base polymer, preferably between 20 and 40 wt % base polymer, more preferably about 30 wt % base polymer. If present, the baseplate precursor solution comprises between 0.1 and 5 wt % additive, preferably between 0.5 and 3 wt %, more preferably about 1.5 wt %.

The solvent is typically water. Other than the glecaprevir and/or pibrentasvir, the components forming the microneedle array are all soluble in water, in addition to its other benefits (e.g. non-toxic, non-flammable, easily available).

The steps of compressing the solutions may use any suitable method known in the art, such as pressure chamber or centrifugation. It is preferred that the step of compressing the microneedle precursor dispersion takes place in a pressure chamber. It is also preferred that compressing the baseplate precursor solution is done by centrifuge.

The drying steps may use any suitable method known in the art. Typically, the drying steps are performed under ambient conditions (i.e. the solvent is simply allowed to evaporate). However, it will be understood that the rate of drying may be increased through the application of increased temperature, increased air flow over the samples, or the application of a reduced pressure.

The microneedle array may retain residual water following drying. The residual water does not exceed 15 wt % of the microneedle array. The residual water content may be between 1 and 15 wt % of the microneedle array, typically between 5 and 10 wt % of the microneedle array.

### Uses of the Solid Compositions, Aqueous Dispersions, Pharmaceutical Compositions, Injectable Formulations, Implantable Rods, and Microneedle Arrays

The present invention provides a solid composition, an aqueous dispersion, a pharmaceutical composition, injectable formulation, implantable rod, or microneedle array as defined herein for use as a medicament.

The present invention provides a solid composition, an aqueous dispersion, a pharmaceutical composition, injectable formulation, implantable rod, or microneedle array as defined herein for use in the treatment and/or prevention of HCV infection and/or Hepatitis C.

In embodiments, the aqueous dispersion, pharmaceutical composition, injectable formulation for use in the treatment and/or prevention of HCV infection and/or Hepatitis

C has a concentration of glecaprevir and/or pibrentasvir in the range of 100 to 1000 mg/mL, preferably 200 to 900 mg/mL, more preferably 300 to 800 mg/mL, and most preferably 500 to 700 mg/mL. Alternatively, the concentration of glecaprevir and/or pibrentasvir is at least 150 mg/mL, preferably at least 200 mg/mL, more preferably at least 300 mg/mL, and most preferably at least 500 mg/mL.

In embodiments, the implantable rod for use in the treatment and/or prevention of HCV infection and/or Hepatitis C has a concentration of glecaprevir and/or pibrentasvir in the range of 40 to 80 wt %, preferably 50 to 70 wt %, most preferably about 60 wt %.

In embodiments, the microneedle array for use in the treatment and/or prevention of HCV infection and/or Hepatitis C contains a mass of glecaprevir and/or pibrentasvir in the range of between 1 and 20 mg of glecaprevir and/or pibrentasvir, preferably between 2 and 10 mg, more preferably about 5 mg. It will be understood that the dose of glecaprevir and/or pibrentasvir provided to a patient may be varied by using larger and/or multiple microneedle arrays.

The present invention provides a method of treating and/or preventing HCV infection and/or Hepatitis C, the method comprising administering a therapeutically effective amount of a solid composition, an aqueous dispersion, a pharmaceutical composition, an injectable formulation, implantable rod, or microneedle array as defined herein to a patient suffering from or at risk of suffering from HCV infection and/or Hepatitis C.

In embodiments, the aqueous dispersion, the pharmaceutical composition, or the injectable formulation has a concentration of glecaprevir and/or pibrentasvir in the range of 100 to 1000 mg/mL, preferably 200 to 900 mg/mL, more preferably 300 to 800 mg/mL, and most preferably 500 to 700 mg/mL. Alternatively, the concentration of glecaprevir and/or pibrentasvir is at least 150 mg/mL, preferably at least 200 mg/mL, more preferably at least 300 mg/mL, and most preferably at least 500 mg/mL.

The aqueous dispersion, pharmaceutical composition, the injectable formulation, or implantable rod may form a depot within the body of the patient, for example, in an intramuscular or subcutaneous site, optionally wherein the depot maintains a therapeutically effective concentration of glecaprevir and/or pibrentasvir within the body of the patient for a period of at least two weeks, preferably at least three weeks, more preferably at least one month, and most preferably at least two months.

Once affixed to the skin, the microneedle array gradually releases glecaprevir and/or pibrentasvir, and optionally maintains a therapeutically effective concentration of glecaprevir and/or pibrentasvir within the body of the patient for a period of at least 4 hours, preferably at least 6 hours, more preferably at least 12 hours, and most preferably at least 24 hours.

In embodiments, the method requires dosing of the aqueous dispersion, the pharmaceutical composition, the injectable formulation, or implantable rod up to three times, preferably up to two times, most preferably only once, to maintain a therapeutically effective concentration of glecaprevir and/or pibrentasvir in the patient for the duration of the treatment.

In embodiments, the method requires dosing of the microneedle array up to six times per day, preferably up to four times per day, more preferably twice a day, and most preferably once a day, to maintain a therapeutically effective concentration of glecaprevir and/or pibrentasvir in the patient for the duration of the treatment.

In any or all of the above-described uses and methods, the administered form of microparticles of glecaprevir and/or pibrentasvir preferably provides a controlled release bolus formulation of glecaprevir and/or pibrentasvir, which, when administered to a patient, releases glecaprevir and/or pibrentasvir into the bloodstream of the patient over a period of at least about two weeks from the date of administration. Further preferably the period of release is at least about three weeks, more preferably at least about one month, most preferably at least about two months from the date of administration of the injection, insertion or application.

### Therapeutic Definitions

As used herein, “treatment” includes curative and prophylactic treatment. As used herein, a “patient” means an animal, preferably a mammal, preferably a human, in need of treatment.

The amount of glecaprevir and/or pibrentasvir administered should be a therapeutically effective amount where glecaprevir and/or pibrentasvir is used for the treatment of a disease or condition and a prophylactically effective amount where the glecaprevir and/or pibrentasvir is used for the prevention of a disease or condition.

The term “therapeutically effective amount” used herein refers to the amount of glecaprevir and/or pibrentasvir needed to treat or ameliorate HCV infection and/or Hepatitis C. The term “prophylactically effective amount” used herein refers to the amount of glecaprevir and/or pibrentasvir needed to prevent HCV infection and/or Hepatitis C. The exact dosage will generally be dependent on the patient's status at the time of administration. Factors that may be taken into consideration when determining dosage include the severity of the disease state in the patient, the general health of the patient, the age, weight, gender, diet, time, frequency and route of administration, drug combinations, reaction sensitivities and the patient's tolerance or response to therapy. The precise amount can be determined by routine experimentation, but may ultimately lie with the judgement of the clinician. An effective dose may in instances be from 0.01 mg/kg/day (mass of drug compared to mass of patient) to 1000 mg/kg/day, e.g. 1 mg/kg/day to 100 mg/kg/day. Compositions may be administered individually to a patient or may be administered in combination with other agents, drugs or hormones. For a long acting injectable, the composition may be administered in an amount sufficient to release glecaprevir and/or pibrentasvir at the above rates. Alternatively, the long acting injectable may be administered in an amount of 0.1 mL to 10 mL, at an amount of 0.2 mL to 6 mL, at an amount of 0.5 mL to 5 mL, or at an amount of 1 mL to 3 mL.

### Routes of Administration

The solid compositions, aqueous dispersions, pharmaceutical compositions, injectable formulations, implantable rods, or microneedle arrays of the invention, may be administered to a patient by any convenient route of administration. More than one route of administration may be used in combination within a defined treatment and/or prophylactic regime, especially for a combination therapy, in which one component of the combination may be administered via one route, whilst another component of the combination may be administered via a different route. All such combinations are hereby contemplated.

Routes of administration include, but are not limited to, oral (e.g. by ingestion); buccal; sublingual; transdermal (including, microneedle array e.g., by a patch, plaster, etc.);

transmucosal (including, e.g., by a patch, plaster, etc.); intranasal (e.g., by nasal spray); ocular (e.g., by eyedrops); pulmonary (e.g., by inhalation or insufflation therapy using, e.g., via an aerosol, e.g., through the mouth or nose); rectal (e.g., by suppository or enema); vaginal (e.g., by pessary); parenteral, for example, by injection, including subcutaneous, intradermal, intramuscular, intravenous, intraarterial, intracardiac, intrathecal, intraspinal, intracapsular, subcapsular, intraorbital, intraperitoneal, intratracheal, subcuticular, intraarticular, subarachnoid, and intrasternal; or by implantation of a depot, reservoir, or implantable rod for example, subcutaneously or intramuscularly.

Most preferably, the route of administration is by injection (e.g. intramuscular or subcutaneous injection) of a depot or implantation of an implantable rod. Alternatively, the route of administration is transdermal via a microneedle array.

Preferably, the injectable formulation of the present invention is a depot formulation administered so as to provide a controlled release in the patient over at least a period of about two weeks from the date of administration. Further preferably the period of release is at least about three weeks, more preferably at least about one month, most preferably at least about two months from the date of administration of the injection.

The implantable rod of the present invention is a depot formulation administered so as to provide a controlled release in the patient over at least a period of about two weeks from the date of administration. Further preferably the period of release is at least about three weeks, more preferably at least about one month, most preferably at least about two months from the date of administration of the injection. Without wishing to be bound by theory, it is thought that the implant gradually dissolves to form a liquid depot and that the glecaprevir and/or pibrentasvir is gradually released from the implant and subsequent liquid depot.

The microneedle array of the present invention is a transdermal release formulation administered to provide a controlled release in a patient over a period of at least four hours, preferably at least 6 hours, more preferably at least 12 hours and most preferably at least 24 hours.

### Kit of Parts

The present invention provides a kit of parts comprising a solid composition as defined herein or pharmaceutical composition comprising the solid composition as defined herein, and a pharmaceutically acceptable aqueous diluent.

The solid composition or pharmaceutical composition comprising the solid composition as defined herein can be dispersed into the diluent to provide an aqueous dispersion as defined herein. Either the entire dispersion can then be administered, or a proportion of it can be measured and then administered (thereby providing a means of administering different dosages to individual patients).

### EXAMPLES

### Materials and Abbreviations

AOT is dioctyl sodium sulfosuccinate.
HPMC is hydroxypropyl methylcellulose.
Kollicoat is a PVA-PEG graft copolymer.
Kollidon 12 PF is a polyvinylpyrrolidone with a K value of around 12.
Kollidon 17 PF is a polyvinylpyrrolidone with a K value of around 17.
Kolliphor EL is a polyethoxylated castor oil formed by the reaction of castor oil with ethylene oxide in a 1:35 molar ratio.
NDC is sodium deoxycholate.
PEG is polyethylene glycol.
Plasdone C15 is a polyvinylpyrrolidone with a K value of around 15.
Plasdone K29/32 is a polyvinylpyrrolidone with a K value between 29 and 32.
Pluronic F68 is poloxamer P188.
Pluronic F127 is poloxamer P407.
PVA is polyvinyl alcohol.
Solutol is polyethylene glycol (15)-hydroxystearate.
Span 20 is sorbitan monolaurate.
Tween 20 is polysorbate 20.
Tween 80 is polysorbate 80.

Example 1: Formation of Pibrentasvir Microparticles by Emulsion Templated Freeze-Drying

Pibrentasvir was dissolved in DCM at a concentration of 60 mg/mL and each of the first and second excipients were dissolved in water at concentrations of 20 mg/mL to form three stock solutions. 100 μL of the pibrentasvir, 150 μL of the first excipient solution, and 50 μL of the second excipient solution were mixed with 200 μL of water to give a mixture with a 1:4 organic solvent to water (O/W) mix. The mixture was emulsified using a Covaris S220x for 30 seconds with a duty cycle of 20, an intensity of 385 and 500 cycles/burst in frequency sweeping mode to achieve an average power output of ˜77 watts. The homogeneous emulsions were immediately frozen by immersion in liquid nitrogen followed by freeze-drying for 48 hours using a VirTis Benchtop Pro with a condenser setting of −100° C. and at a pressure of <40 μBar.

The resulting solid product was in the form of a monolith containing 60 wt % pibrentasvir, 30 wt % of the first excipient and 10 wt % of the second excipient. The first and second excipients used are listed in the table below.

| 1^{st} excipient | 2^{nd} excipient |
|---|---|
| Kolliphor EL | Benzalkonium chloride |
| Plasdone C15 | AOT |
| Lactose | Pluronic F68 |
| Sucrose | Tween 20 |
| Pluronic F68 | Tween 80 |
| PEG 4000 | Span 20 |

Each solid composition was dispersed in water to give a microparticulate dispersion with a pibrentasvir concentration of 1.5 mg/mL. The particle diameter of each dispersion was measured by dynamic light scattering (DLS—sometimes referred to as Photon Correlation Spectroscopy (PCS)) using a Zetasizer Ultra available from Malvern Panalytical Limited. The measurements were performed in triplicate, the instrument operating at a temperature of 25° C. and a measurement angle of 173° (backscatter). Data analysis was conducted using the general-purpose model within the ZS Xplorer software. The combinations of first and second excipients were overall considered to be “hits” if the resulting dispersions had a Z-average hydrodynamic diameter (Dz), measured in at least one experiment, of less than 3000 nm, preferably less than 2600 nm, and a PDI value of less than 0.5.

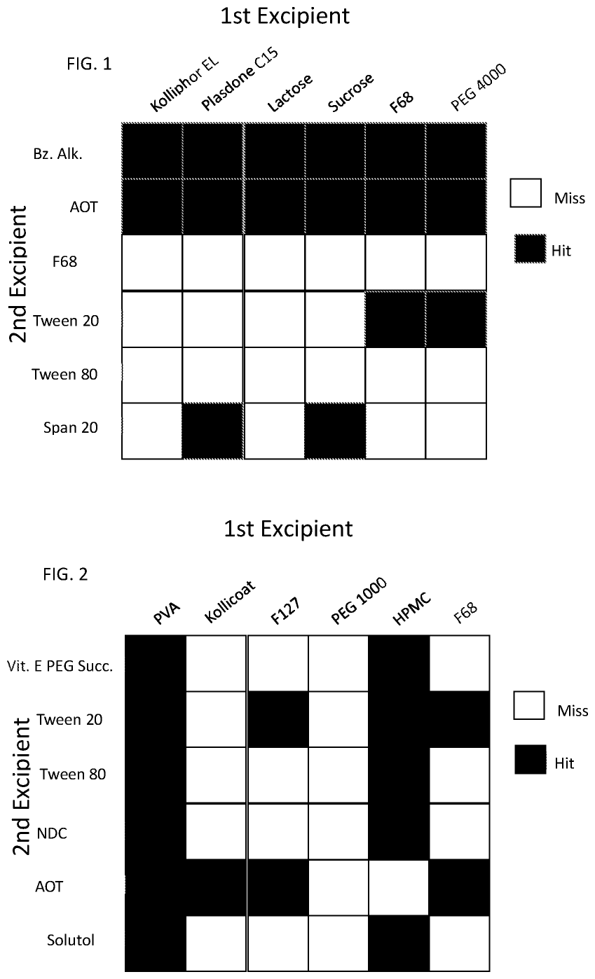

The results of the DLS measurements are shown in FIG. 1, with the details of the highest quality dispersions tabulated below.

| | | | | Data |
|---|---|---|---|---|
| 1st Excipient | 2nd Excipient | Dz ± σ (nm) | PDI ± σ | Quality |
| Plasdone C15 | Benzalkonium | 1150 ± 75 | 0.398 ± 0.089 | Good |
| | chloride | | | |
| | AOT | 1410 ± 85 | 0.362 ± 0.036 | Good |
| Lactose | Benzalkonium | 1630 ± 185 | 0.357 ± 0.071 | Good |
| | chloride | | | |
| Sucrose | AOT | 1900 ± 70 | 0.301 ± 0.073 | Good |
| Pluronic F68 | Benzalkonium | 2030 ± 65 | 0.257 ± 0.042 | Good |
| | chloride | | | |
| | Tween 20 | 1720 ± 50 | 0.156 ± 0.024 | Good |
| PEG 4000 | Benzalkonium | 2190 ± 60 | 0.223 ± 0.027 | Good |
| | chloride | | | |

The syringeability of each composition was tested at increasing concentrations of pibrentasvir. The dispersions were produced by vortex mixing the solid composition in water at the required concentrations for a period of 30 seconds. The dispersions were then passed through a 25G needle by hand. Those that passed through easily and without blockages, with a repeat one hour later, were considered to have passed. The pibrentasvir₆₀/Plasdone C15₃₀/AOT₁₀ dispersions were found to be syringeable at all tested concentrations up to the highest tested concentration of 200 mg/mL with respect to pibrentasvir.

### Example 2: Formation of Pibrentasvir Microparticles by Emulsion Templated Freeze-Drying

The methodology of Example 1 using DCM as the organic solvent was followed to produce pibrentasvir compositions comprising 60 wt % pibrentasvir, 30 wt % first excipient and 10 wt % second excipient, with the excipients being those listed in the Table below.

| 1^{st} excipient | 2^{nd} excipient |
|---|---|
| PVA | Solutol |
| Kollicoat | AOT |
| Pluronic F127 | NDC |
| PEG 1000 | Tween 20 |
| HPMC | Tween 80 |
| Pluronic F68 | Vit-E-PEG succinate |

The solid compositions were dispersed in water to give microparticulate dispersions with pibrentasvir concentrations of 1.5 mg/mL. The particle diameter of the dispersion was assessed as in Example 1. The results are summarised in FIG. 2, with the details of the highest quality dispersions tabulated below.

| 1st Excipient | 2nd Excipient | Dz (nm) | PDI | Data Quality |
|---|---|---|---|---|
| PVA | Vit-E-PEG succinate | 660 ± 4 | 0.266 | Good |
| | Tween 20 | 420 ± 5 | 0.175 | Good |
| | Tween 80 | 550 ± 18 | 0.247 | Good |
| | NDC | 590 ± 10 | 0.328 | Good |
| | Solutol | 660 ± 4 | 0.232 | Good |
| Kollicoat | AOT | 480 ± 7 | 0.409 | Good |
| HPMC | Vit-E-PEG succinate | 1240 ± 9 | 0.266 | Good |
| | Tween 20 | 1270 ± 45 | 0.242 | Good |
| | Tween 80 | 1460 ± 23 | 0.250 | Good |
| | NDC | 1800 ± 91 | 0.216 | Good |

The experiment was repeated to provide pibrentasvir compositions comprising 70 wt % pibrentasvir, 20 wt % first excipient and 10 wt % second excipient. The details of the highest quality dispersions are tabulated below.

| 1st Excipient | 2nd Excipient | Dz (nm) | PDI | Data Quality |
|---|---|---|---|---|
| PVA | Vit-E-PEG succinate | 730 ± 25 | 0.284 | Good |
| | Tween 20 | 580 ± 20 | 0.241 | Good |
| | Tween 80 | 660 ± 5 | 0.271 | Good |
| | NDC | 720 ± 20 | 0.478 | Good |
| | AOT | 1200 ± 80 | 0.656 | Good |
| | Solutol | 630 ± 30 | 0.248 | Good |
| HPMC | Vit-E-PEG succinate | 1260 ± 40 | 0.226 | Good |
| | Tween 20 | 1270 ± 50 | 0.301 | Good |
| | Tween 80 | 1530 ± 125 | 0.292 | Good |
| | Solutol | 1490 ± 30 | 0.302 | Good |

### Example 3: Formation of Pibrentasvir Microparticles by Emulsion Templated Freeze-Drying

The process for preparing the solid compositions of Example 1 was repeated with the same first and second excipients, using ethyl acetate as the solvent in place of DCM.

Each solid composition was dispersed in water to give a microparticulate dispersion with a pibrentasvir concentration of 1.5 mg/mL. The particle diameter of each dispersion was assessed as in Example 1.

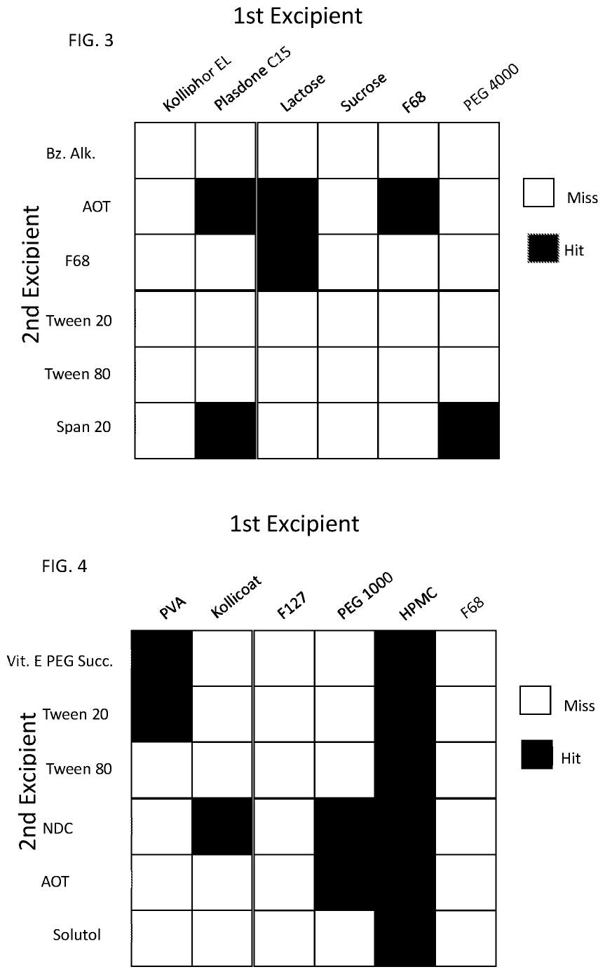

The results of the DLS measurements are shown in FIG. 3, with the details of the highest quality dispersions tabulated below.

| 1st | 2nd | Dz ± σ | | Data |
|---|---|---|---|---|
| Excipient | Excipient | (nm) | PDI ± σ | Quality |
| Plasdone C15 | AOT | 1150 ± 10 | 0.278 ± 0.034 | Good |
| Pluronic F68 | AOT | 1450 ± 105 | 0.312 ± 0.054 | Good |

The syringeability of each dispersion was assessed as in Example 1. The pibrentasvirso/Plasdone C15₃₀/AOT₁₀ dispersion was found to be syringeable at all tested concentrations up to the highest tested concentration of 200 mg/mL with respect to pibrentasvir.

### Example 4: Formation of Pibrentasvir Microparticles by Emulsion Templated Freeze-Drying

The process for preparing the solid compositions of Example 2 was repeated with the same first and second excipients, using ethyl acetate as the solvent in place of DCM.

Each solid composition was dispersed in water to give a microparticulate dispersion with a pibrentasvir concentration of 1.5 mg/mL. The particle diameter of each dispersion was assessed as in Example 1.

The results of the DLS measurements are shown in FIG. 4, with the details of the highest quality dispersions tabulated below.

| 1st | 2nd | | | Data |
|---|---|---|---|---|
| Excipient | Excipient | Dz (nm) | PDI | Quality |
| PVA | Vit-E-PEG succinate | 780 ± 25 | 0.471 | Good |
| | Tween 20 | 710 ± 5 | 0.476 | Good |
| Kollicoat | NDC | 1280 ± 90 | 0.366 | Good |
| PEG | NDC | 1940 ± 1000 | 0.332 | Good |
| | AOT | 1730 ± 40 | 0.252 | Good |
| HPMC | Vit-E-PEG succinate | 1450 ± 60 | 0.377 | Good |
| | Tween 20 | 1320 ± 30 | 0.318 | Good |
| | Tween 80 | 1300 ± 60 | 0.364 | Good |
| | NDC | 1610 ± 20 | 0.387 | Good |
| | AOT | 1510 ± 160 | 0.317 | Good |
| | Solutol | 1800 ± 50 | 0.171 | Good |

The experiment was repeated to provide pibrentasvir compositions comprising 70 wt % pibrentasvir, 20 wt % first excipient and 10 wt % second excipient. The details of the highest quality dispersions are tabulated below.

| 1st Excipient | 2nd Excipient | Dz (nm) | PDI | Data Quality |
|---|---|---|---|---|
| HPMC | Tween 20 | 1310 ± 20 | 0.223 | Good |
| | Tween 80 | 1290 ± 90 | 0.260 | Good |
| | NDC | 2080 ± 70 | 0.311 | Good |
| | AOT | 1810 ± 50 | 0.285 | Good |
| | Solutol | 1870 ± 20 | 0.176 | Good |

### Example 5: Formation of Pibrentasvir Microparticles by Emulsion Spray-Drying

One of the promising candidates from the foregoing examples, pibrentasvirs₆₀/Plasdone C15₃₀/AOT₁₀, was selected for scaled up production via emulsion spray-drying.

Pibrentasvir was dissolved in DCM at a concentration of 171.4 mg/mL, Plasdone C15 was dissolved in water at a concentration of 40 mg/mL, and AOT was dissolved in water at concentrations of 10 mg/mL to form three stock solutions.

Plasdone C15 (1.5 mL), AOT (2 mL) and DI-H₂O (5.8 mL) were added to a 14 mL vial followed by pibrentasvir (0.7 mL). The vial contents was then sonicated for 15 seconds using a Hielscher probe sonicator with an H7 attachment (cycle 1, amplitude 100%) to generate an emulsion, which was then immediately spray-dried at a flow-rate of 5 mL/min (Buchi Mini B-290: aspirator 100%, nitrogen (5 bar pressure), Q-flow gauge 45, outlet temperature 65° C.).

The solid compositions were dispersed in water to give microparticulate dispersions with pibrentasvir concentrations of 1 mg/mL. The particle diameter of the dispersion was assessed as in Example 1. Repeats of the production process demonstrated that the method produces nanoparticles with high reproducibility.

| Formulation Repeat | Dz ± σ (nm) | PDI ± σ |
|---|---|---|
| 1 | 206 ± 6 | 0.349 ± 0.005 |
| 2 | 200 ± 4 | 0.331 ± 0.009 |
| 3 | 209 ± 3 | 0.363 ± 0.019 |
| 4 | 218 ± 9 | 0.398 ± 0.043 |
| 5 | 200 ± 2 | 0.292 ± 0.01 |

The syringeability of each dispersion was assessed as in Example 1. The pibrentasvirso₆₀/Plasdone C15₃₀/AOT₁₀ dispersion was found to be syringeable at all tested concentrations up to the highest tested concentration of 500 mg/mL with respect to pibrentasvir.

### Example 6: Formation of Glecaprevir Microparticles by Emulsion Templated Freeze-Drying

The methodology of Example 1 using DCM as the organic solvent was followed to produce glecaprevir compositions comprising 70 wt % glecaprevir, 24 wt % first excipient and 6 wt % second excipient, with the excipients being those listed in Example 2.

The solid compositions were dispersed in water to give microparticulate dispersions with glecaprevir concentrations of 1 mg/mL. The particle diameter of the dispersion was assessed as in Example 1. The results are summarised in FIG. 5, with the details of the highest quality dispersions tabulated below.

| 1st Excipient | 2nd Excipient | Dz (nm) | PDI | Data Quality |
|---|---|---|---|---|
| Kollicoat | Solutol | 891 | 0.307 | Good |
| | AOT | 715 | 0.454 | Good |
| PVA | Solutol | 745 | 0.270 | Good |
| | AOT | 1034 | 0.397 | Good |
| | NDC | 1122 | 0.368 | Good |
| | Tween 20 | 712 | 0.349 | Good |
| | Tween 80 | 728 | 0.340 | Good |
| | Vit-E-PEG succinate | 815 | 0.349 | Good |

### Example 7: Formation of Glecaprevir Microparticles by Emulsion Templated Freeze-Drying

The methodology of Example 3 using ethyl acetate as the organic solvent was followed to produce Glecaprevir compositions comprising 70 wt % glecaprevir, 20 wt % first excipient and 10 wt % second excipient, with the excipients being those listed in Example 2.

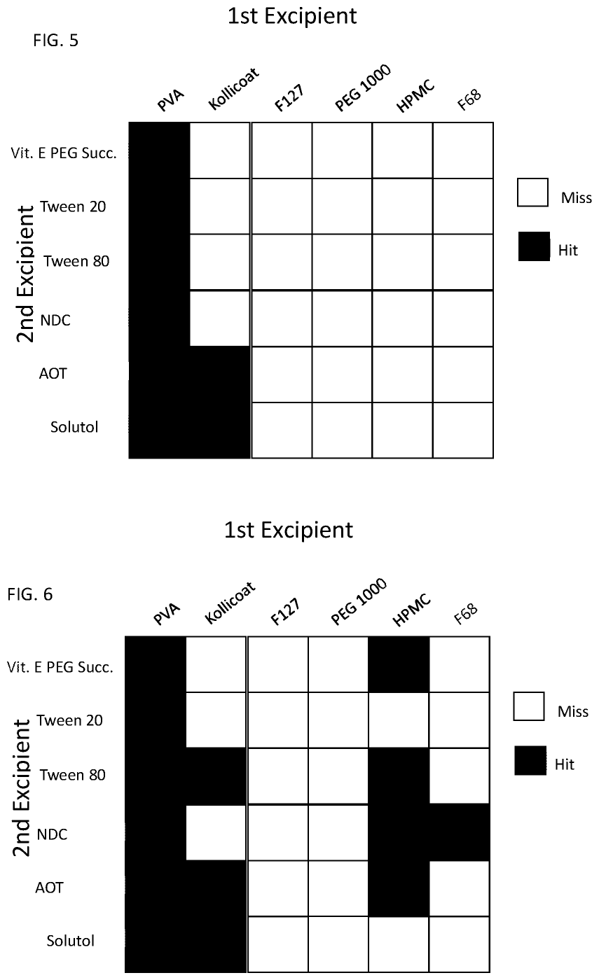

The solid compositions were dispersed in water to give microparticulate dispersions with glecaprevir concentrations of 2 mg/mL. The particle diameter of the dispersion was assessed as in Example 1. The results are summarised in FIG. 6, with the details of the highest quality dispersions tabulated below.

| 1st Excipient | 2nd Excipient | Dz (nm) | PDI | Data Quality |
|---|---|---|---|---|
| Kollicoat | Solutol | 926 | 0.437 | Good |
| | Tween 80 | 886 | 0.461 | Good |
| PVA | Solutol | 824 | 0.366 | Good |
| | NDC | 706 | 0.302 | Good |
| | Tween 20 | 675 | 0.235 | Good |
| | Tween 80 | 708 | 0.346 | Good |
| | Vit-E-PEG succinate | 707 | 0.274 | Good |

### Example 8: Formation of Glecaprevir Microparticles by Emulsion Templated Freeze-Drying

The process for preparing the solid compositions of Example 1 was repeated with the first and second excipients listed in the table below, to produce solid compositions comprising 70 wt % glecaprevir, 20 wt % first excipient, and 10 wt % second excipient, with DCM as the organic solvent.

| 1^{st} excipient | 2^{nd} excipient |
|---|---|
| Kolliphor EL | Benzalkonium chloride |
| Plasdone C15 | AOT |
| Kollidon PF17 | Pluronic F127 |
| Lactose | Tween 20 |
| Sucrose | Tween 80 |
| Pluronic F127 | Span 20 |

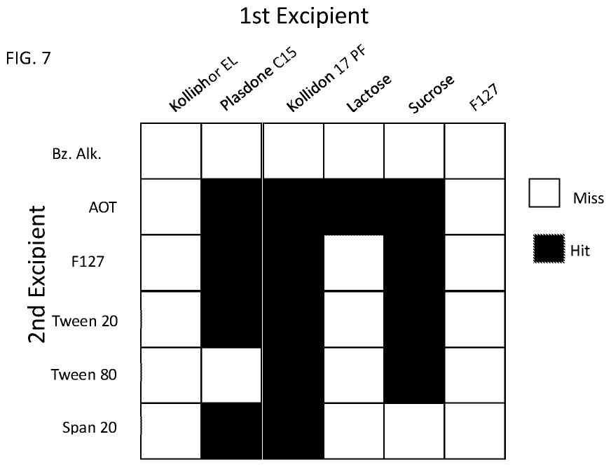

The solid compositions were dispersed in water to give microparticulate dispersions with glecaprevir concentrations of 2 mg/mL. The particle diameter of the dispersion was assessed as in Example 1. The results are summarised in FIG. 7, with the details of the highest quality dispersions tabulated below.

| 1st Excipient | 2nd Excipient | Dz (nm) | PDI | Data Quality |
|---|---|---|---|---|
| Plasdone C15 | AOT | 1706 | 0.656 | Good |
| Plasdone C15 | Pluronic F127 | 916 | 0.491 | Good |
| Plasdone C15 | Tween 20 | 844 | 0.390 | Good |
| Plasdone C15 | Span 20 | 1163 | 0.398 | Good |
| Kollidon 17 PF | AOT | 1335 | 0.489 | Good |
| Kollidon 17 PF | Pluronic F127 | 1592 | 0.413 | Good |
| Kollidon 17 PF | Tween 20 | 1319 | 0.341 | Good |
| Kollidon 17 PF | Tween 80 | 1593 | 0.342 | Good |
| Lactose | AOT | 1425 | 0.369 | Good |
| Sucrose | AOT | 1283 | 0.651 | Good |
| Sucrose | Pluronic F127 | 1655 | 0.371 | Good |
| Sucrose | Tween 20 | 1869 | 0.322 | Good |
| Sucrose | Tween 80 | 1826 | 0.388 | Good |

### Example 9: Formation of Glecaprevir Microparticles by Emulsion Templated Freeze-Drying

The process of Example 8 was repeated, varying the loading of glecaprevir, first excipient, and second excipient within the solid composition, as well as testing with alternative PVPs.

In a first test, compositions were produced comprising 50 wt % glecaprevir, 33 or 40 wt % first excipient, and 17 or 10 wt % second excipient. The first excipient was a PVP selected from one of Plasdone C15, Kollidon 12 PF, and Kollidon 17 PF. The second excipient was AOT. Each of the compositions was dispersed in water and the resulting dispersion tested by DLS.

| 1st Excipient | 2nd Excipient | Dz (nm) | PDI | Data Quality |
|---|---|---|---|---|
| 33 wt % Plasdone C15 | 17 wt % AOT | 1057 | 0.244 | Good |
| 33 wt % Kollidon 12 PF | 17 wt % AOT | 1465 | 0.401 | Good |
| 33 wt % Kollidon 17 PF | 17 wt % AOT | 1291 | 0.52 | Good |
| 40 wt % Plasdone C15 | 10 wt % AOT | 1341 | 0.318 | Good |
| 40 wt % Kollidon 12 PF | 10 wt % AOT | 1136 | 0.283 | Good |
| 40 wt % Kollidon 17 PF | 10 wt % AOT | 1079 | 0.417 | Good |

In a second test, compositions were produced comprising 70 wt % glecaprevir, 27, 24, or 6 wt % first excipient, and 3, 6, or 24 wt % second excipient. The first excipient was a PVP selected from one of Plasdone C15, Kollidon 12 PF, and Kollidon 17 PF, while the second excipient was one of AOT, Tween 20, and Tween 80. Each of the compositions was dispersed in water and the resulting dispersion tested by DLS.

| | | Dz | | Data |
|---|---|---|---|---|
| 1st Excipient | 2nd Excipient | (nm) | PDI | Quality |
| 27 wt % Plasdone C15 | 3 wt % AOT | 769 | 0.532 | Good |
| 27 wt % Kollidon 12 PF | 3 wt % Tween 20 | 970 | 0.425 | Good |
| 27 wt % Kollidon 17 PF | 3 wt % Tween 80 | 1124 | 0.482 | Good |
| 24 wt % Plasdone C15 | 6 wt % AOT | 1122 | 0.448 | Good |
| 24 wt % Kollidon 17 PF | 6 wt % Tween 80 | 1225 | 0.576 | Good |
| 6 wt % Plasdone C15 | 24 wt % AOT | 1618 | 0.472 | Good |
| 6 wt % Kollidon 12 PF | 24 wt % Tween 20 | 1343 | 0.206 | Good |
| 6 wt % Kollidon 17 PF | 24 wt % Tween 80 | 1618 | 0.386 | Good |

In a third test, compositions were produced comprising 80 wt % glecaprevir, 18 wt % first excipient and 2 wt % second excipient. The first excipient was a PVP selected from one of Plasdone C15, Kollidon 12 PF, and Kollidon 17 PF. The second excipient was AOT. The syringability of each of these compositions was tested, with each being syringable at all tested concentrations up to the highest tested concentration of 250 mg/mL with respect to glecaprevir.

### Example 10: Formation of Glecaprevir Microparticles by Emulsion Spray-Drying

The spray drying methodology of Example 5 was followed to produce the following solid compositions:
Glecaprevir₆₀/Plasdone C15₃₀/AOT₁₀
Glecaprevir₆₀/Kollidon 12PF₃₀/AOT₁₀
Glecaprevir₆₀/Kollidon 17PF₃₀/AOT₁₀

Each of the solid compositions was dispersed in water and the resulting dispersion analysed as set out in Example 5. Each of the compositions was found to produce high quality dispersion, the characteristics of which are tabulated below.

| 1st Excipient | 2nd Excipient | Dz (nm) | PDI | Data Quality |
|---|---|---|---|---|
| 30 wt % Plasdone C15 | 10 wt % AOT | 183 | 0.223 | Good |
| 30 wt % Kollidon 12 PF | 10 wt % AOT | 279 | 0.472 | Good |
| 30 wt % Kollidon 17 PF | 10 wt % AOT | 195 | 0.241 | Good |

In addition, the syringability of each of these compositions was tested, with each being syringable at all tested concentrations up to the highest tested concentration of 650 mg/mL with respect to glecaprevir.

### Example 11: Formation of Glecaprevir and Pibrentasvir Microparticles by Emulsion Spray-Drying

The spray drying methodology of Example 5 was followed to produce compositions comprising both glecaprevir and pibrentasvir. Each of the below formulations comprised (glecaprevir and pibrentasvir) 60/Plasdone C15₃₀/AOT₁₀, with the mass ratio of glecaprevir to pibrentasvir being varied. Each of the solid compositions was dispersed in water and the resulting dispersion analysed as set out in Example 5. Each of the compositions was found to produce high quality dispersion, the characteristics of which are tabulated below.

| Glecaprevir:Pibrentasvir | Dz | | Data |
|---|---|---|---|
| (mass ratio) | (nm) | PDI | Quality |
| 1:3 | 190 | 0.272 | Good |
| 1:1 | 179 | 0.239 | Good |
| 3:1 | 169 | 0.177 | Good |

In addition, the syringability of 1:1 composition (i.e. the glecaprevir₃₀/pibrentasvir₃₀/Plasdone C15₃₀/AOT₁₀) was tested, and the composition was found to be syringable at all tested concentrations up to the highest tested concentration of 500 mg/mL with respect to glecaprevir and pibrentasvir combined.

### Example 12: Formation of Glecaprevir and Pibrentasvir Microparticles by Emulsion Spray-Drying using Different Solvents

The spray drying methodology of Example 5 was followed to produce compositions comprising both glecaprevir and pibrentasvir. Each of the two formulations below comprised glecaprevir₃₀/pibrentasvir₃₀/Plasdone C15₃₀/AOT₁₀, with the organic solvent being either anisole or ethyl acetate.

| Solvent | Repeat | Dz (nm) | PDI |
|---|---|---|---|
| Anisole | 1 | 1186 | 0.224 |
| | 2 | 1174 | 0.194 |
| | 3 | 1247 | 0.124 |
| Ethyl | 1 | 545 | 0.639 |
| acetate | 2 | 566 | 0.678 |
| | 3 | 689 | 0.633 |

In addition, the syringability of each of the compositions was tested and found to be syringable at all tested concentrations up to the highest tested concentration of 500 mg/mL with respect to glecaprevir and pibrentasvir combined.

### Example 13: Formulation into Implantable Rods

Implantable rods were prepared by a vacuum compression moulding (VCM) method using a MeltPrep VCM Essentials instrument set-up consisting of a hot plate, nitrogen gas assisted cooling plate, vacuum pump, base plate, VCM sample chamber, VCM main body, 2 mm internal diameter PTFE sample tube, 2 mm diameter PTFE-coated separation foils, 15 mm piston, and a low-pressure lid.

Prior to sample preparation, the hot-plate was heated to a temperature of 110° C. and a vacuum pressure of −1 bar was maintained for approximately 20 minutes.

The sample tube was inserted into the VCM sample chamber, which was then fitted onto the base plate. A separation foil was then inserted and positioned at the bottom of the tube before adding the powdered formulation (˜45 mg) using a funnel, which was compacted as much as possible using a pin. A second separation foil was then positioned on top of the sample before inserting a 15 mm piston into the sample tube. The VCM main body was then positioned over this assembly before attaching the low-pressure lid. A vacuum of −1 barg was applied to the sample chamber before placing it on the hot plate. The sample was heated to 110° C. for 10 minutes before being transferred onto the cooling plate and cooled for 15 minutes. Translucent, yellow coloured rods were obtained weighing ˜45 mg and having a length of 11 mm and diameter of 2 mm.

### Example 14: In vivo Longevity of Pibrentasvir/Glecaprevir following Intramuscular Injection at Varying Volume

An aqueous dispersion of the glecaprevir₃₀/pibrentasvir₃₀/Plasdone C15₃₀/AOT₁₀ formulation in water was produced with a total drug concentration of 500 mg/mL.

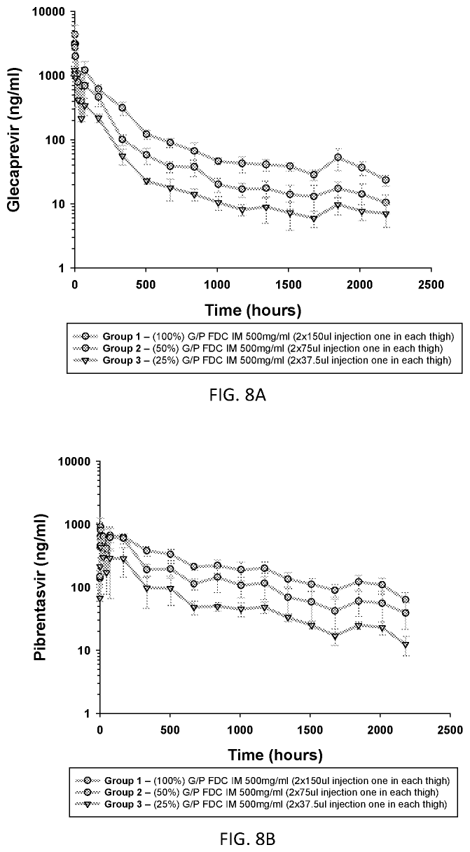

Male Sprague Dawley Rats (250-300 g-Envigo) were injected intramuscularly into both thighs with different volumes (150 μl, 75 μl or 37.5 μl per thigh) of the stock solution for each group of four animals. Blood plasma was collected from the tail veins periodically over the course of 91-days and the glecaprevir/pibrentasvir concentrations therein quantified using LC/MS-MS. This data is graphed in FIGS. 8A and 8B and shows a gradual decline in the concentration of glecaprevir and pibrentasvir over the 91-day period of the experiment, demonstrating that relevant concentrations of glecaprevir and pibrentasvir are maintained for the duration.

### Example 15: In vivo Longevity of Pibrentasvir/Glecaprevir following Intramuscular Injection at Constant Volume

Aqueous dispersions of the glecaprevir₃₀/pibrentasvir₃₀/Plasdone C15₃₀/AOT₁₀ formulation in water were produced with a total drug concentration of 500 mg/mL, 250 mg/mL, and 125 mg/mL.

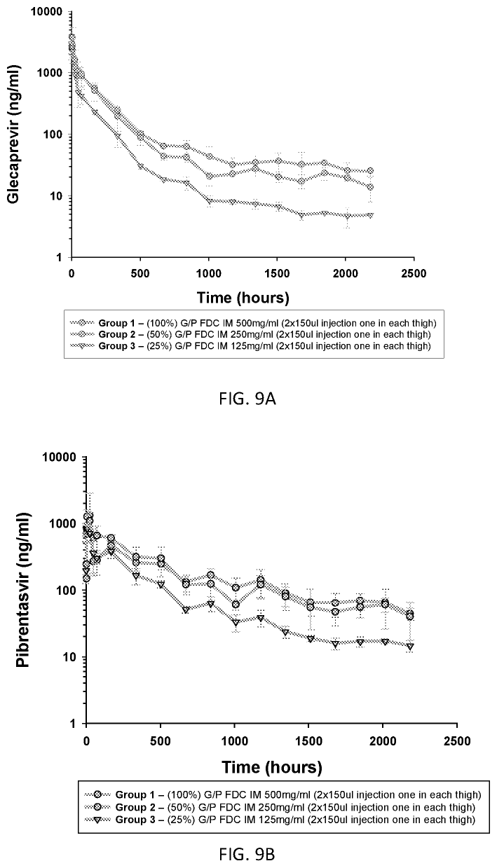

Male Sprague Dawley Rats (250-300 g-Envigo) were injected intramuscularly into both thighs with 150 μl of each the stock solutions with different concentration for each group of four animals. Blood plasma was collected from the tail veins periodically over the course of 2200 hours and the glecaprevir/pibrentasvir concentrations therein quantified using LC/MS-MS. This data is graphed in FIGS. 9A and 9B and shows a gradual decline in the concentration of glecaprevir and pibrentasvir over the 91-day period of the experiment, demonstrating that relevant concentrations of glecaprevir and pibrentasvir are maintained for the duration.

### Example 16: In vivo Longevity of Pibrentasvir/Glecaprevir following Implantation of a Rod

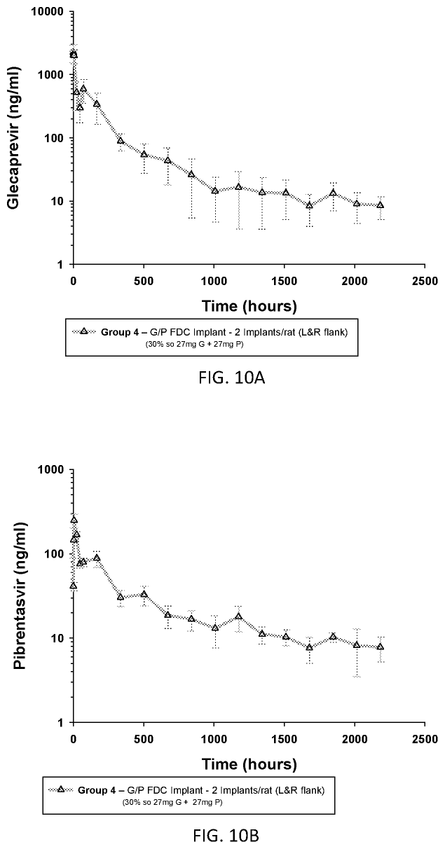

This experiment was performed on four male Sprague Dawley Rats (250-300 g-Envigo). Each rat was implanted with two 11 mm rods, the rods implanted subcutaneously into each scapular region. Each rod had a mass of approximately 45 mg, providing each rat with a total dose of approximately 27 mg glecaprevir and 27 mg pibrentasvir. Blood plasma was collected from the tail veins periodically over the course of 2200 hours and the glecaprevir/pibrentasvir concentrations therein quantified using LC/MS-MS. This data is graphed in FIGS. 10A and 10B and shows a gradual decline in the concentration of glecaprevir and pibrentasvir over the 91-day period of the experiment, demonstrating that relevant concentrations of glecaprevir and pibrentasvir are maintained for the duration.

### Example 17: In vivo Longevity of Glecaprevir, Pibrentasvir, and combined Glecaprevir/Pibrentasvir following Intramuscular Injection

Three aqueous dispersions in saline were formed using glecaprevirco/Plasdone C15₃₀/AOT₁₀, pibrentasvir60/Plasdone C15₃₀/AOT₁₀, or glecaprevir₃₀/pibrentasvir₃₀/Plasdone C15₃₀/AOT₁₀, each with a total drug concentration of 500 mg/mL.

Male Sprague Dawley Rats (250-300 g-Envigo) were injected intramuscularly into each thighs with 150 μl of the stock solutions as follows:
Group 1—Glecaprevir dispersion in each thigh;
Group 2—Pibrentasvir dispersion in each thigh;
Group 3—Glecaprevir dispersion in the left thigh and Pibrentasvir solution in the right thigh; and
Group 4—Mixed Glecaprevir and Pibrentasvir dispersion in each thigh.

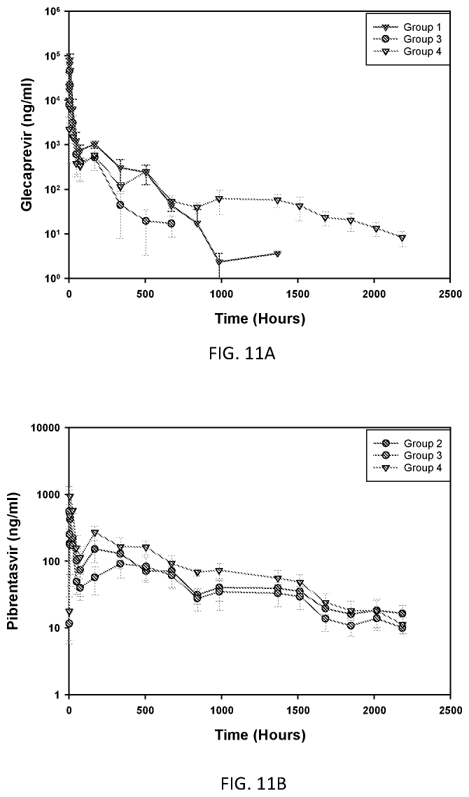

Each group contained four animals. Blood plasma was collected from the tail veins periodically over the course of 2200 hours and the glecaprevir/pibrentasvir concentrations therein quantified using LC/MS-MS. This data is graphed in FIGS. 11A and 11B and shows a gradual decline in the concentration of glecaprevir and pibrentasvir over the 91-day period of the experiment.

### Example 18: Liver: plasma ratio for Glecaprevir and Pibrentasvir

An aqueous dispersion of the glecaprevir₃₀/pibrentasvir₃₀/Plasdone C15₃₀/AOT₁₀ formulation in water was produced with a total drug concentration of 500 mg/mL.

Male Sprague Dawley Rats (250-300 g-Envigo) were injected intramuscularly into both thighs with 150 μl per thigh of the stock solution for each group of four animals. After 91 days, the animals were sacrificed and the livers and blood plasma were assessed to determine the ratio of liver to plasma concentration of each drug. These results were compared to known literature values for orally administered Maviret (for glecaprevir: EMA Assessment Report EMA/449689/2017; for pibrentasvir: Australian Public

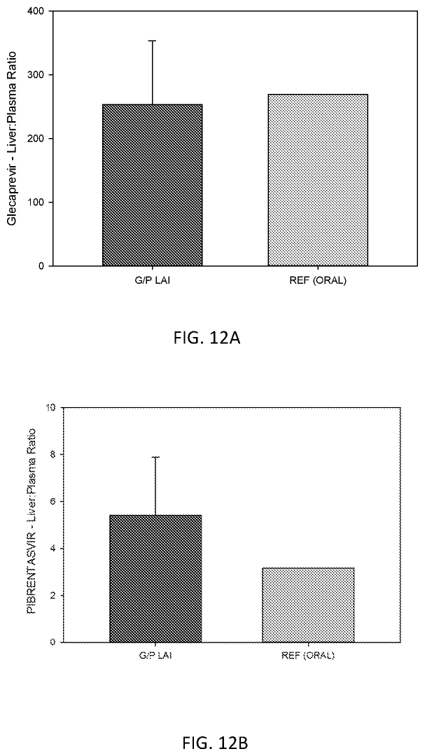

Assessment Report for Glecaprevir/pibrentasvir) to determine if the absence of first pass metabolism in the liver would decrease the concentration of each drug in the liver, the target tissue for treating HCV. As can be seen in FIGS. 12A and 12B, it was found that the intramuscularly dosed glecaprevir and pibrentasvir had very similar liver distribution to the orally dosed Maviret.

### Example 19: Formulation into Microneedle Arrays

A polymer stock solution was prepared containing 20% w/w PVA (Sigma Aldrich, nominal MW of 9-10 kDa, Mw 9-10 kDa) and 20% w/w Plasdone K29/32 in deionised water. A needle layer composition was prepared by mixing 25% w/w of the polymer stock solution with 31.25% w/w of the glecaprevir₃₀/pibrentasvir₃₀/Plasdone C15₃₀/AOT₁₀ formulation and 43.75% w/w of deionised water using a Speedmixer™ at 3500 rpm for 5 minutes. A baseplate composition was prepared by mixing 30%/w PVP

(Sigma Aldrich, nominal MW of 360 kDa, Mn 360 kDa, K Value 80-100), 1.5% w/w glycerol and balance deionised water prior to sonication and centrifugation.

The needle layer composition was cast into a 16 by 16 array arranged on a 0.49 cm² area, each needle having a height of 850 μm (of which 600 μm is pyramidal tip and 250 um is base column) and a column width of 300 μm, the spacing between needles being 100 μm. The array was placed into a pressure chamber and subjected to a pressure of 5 bar for 3 minutes. In some embodiments a second needle layer is cast and the array is placed in the pressure chamber and subjected to a pressure of 5 bar for a further 5 minutes. The microneedles were left to dry overnight.

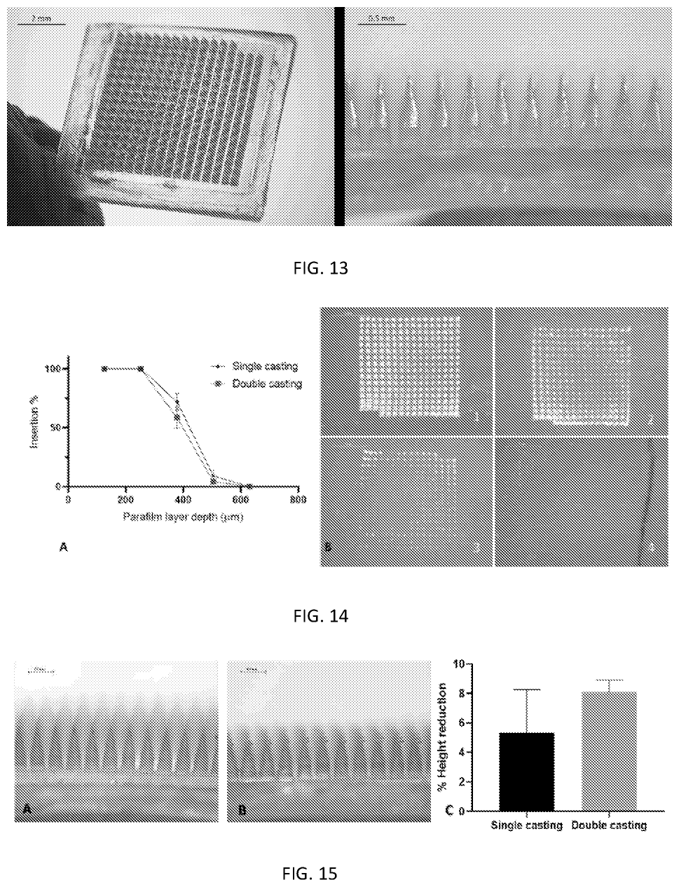

650 μl of baseplate composition was cast onto the prepared needles and the arrays centrifuged for 15 minutes as 5000 rpm before drying under ambient conditions for 48 hours. The set microneedle arrays were then released from the moulds and excess baseplate material cut away. Plan and profile views of a typical microneedle array produced by the above process are shown in FIG. 13.

### Example 20: Physical Characterisation of the Microneedle Arrays

The insertion efficiency, compressive strength, and drug loading of the microneedle arrays were determined.

For the insertion study, the microneedle arrays were applied to layered Parafilm® M (each layer having a thickness of approximately 252 μm) as an in vitro skin model using 32 N of force (equivalent to that of a human thumb). The number of holes punctured in each layer of Parafilm® M was used to calculate the % insertion for each layer using the following equation:

$$Insertion \% = \frac{number of holes observed}{number of needles on array} \times 100$$

Puncture marks made in four successive layers of Parafilm® M are shown for one example in FIG. 14, which also shows the results comparing the insertion efficiency of the single and double casting of the microneedle composition. It can be seen that the two castings have similar insertion efficiencies, with each completely penetrating the first two layers of Parafilm® M and maintaining some penetration through to the fourth layer of Parafilm® M.

For compression testing, the heights of 20 microneedles (five from each edge of a microneedle array) were visually measured under a light microscope to provide HO. 32N of force was applied to each patch for 30 seconds and then the heights of the microneedles were remeasured as before to provide H1.

$$Height Reduction \% = \frac{H 0 - H 1}{H 0} \times 100$$

Profile micrographs of a typical microneedle array before and after application of the 32 N force are shown in FIG. 15, along with a graph of the average height reduction % before and after application of the 32 N force. For both the single and double-cast microneedles, the height reduction was less than 10% and the difference between the castings was not significant. Furthermore, examination of the microneedles following compression showed that the microneedles are bending, not breaking, indicating that they can be safely inserting into the skin.

To quantify the drug loading of the microneedle arrays, three singly cast microneedle arrays were dissolved in 5 ml of water and sonicated for 30 minutes to ensure complete dissolution, followed by the addition of 5 mL of methanol and dilution by a factor of 10. The glecaprevir and pibrentasvir content was determined using HPLC. The process was repeated for the doubly-cast microneedle arrays.

| Mass of drug per | | |
|---|---|---|
| microarray (mg) | Glecaprevir | Pibrentasvir |
| Singly-cast | 1.46 ± 0.29 | 1.32 ± 0.26 |
| Doubly-cast | 1.95 ± 0.23 | 1.79 ± 0.20 |

As expected, the doubly-cast samples had a higher loading of each drug.

### Example 21: Ex vivo Assessment of the Microneedles

Ex vivo skin deposition experiments were carried out utilising a modified Franz diffusion cell. Full thickness skin was collected and excised within 24 hours of birth. On the day of the experiment, the skin was first pre-equilibrated in PBS at pH 7.4 for 30 minutes until totally thawed and then carefully shaved using a razor. It was then cyanoacrylate-glued to the donor compartment of the Franz diffusion cells to ensure its adhesion to the set up during the microneedle insertion and throughout the experiment. Microneedle arrays were applied to the skin using firm thumb pressure for 30 seconds. A stainless-steel cylinder (diameter 11 mm, mass 11.5 g) was put on the top of each microneedle array to hold them in place throughout the experiment.

The receiver compartment was filled with 12 mL of 1% w/v SLS in PBS (pH 7.4). The donor compartment was clamped on top of the receiver compartment and wrapped with Parafilm® M to prevent solvent evaporation. Samples of 200 μL of the receiver compartment were taken at predefined time points of 0.5, 1, 1.5, 2, 3, 4, 6, 10, and 24 hours and was replaced with the fresh release medium. At 24 hours, the Franz cells were disassembled, the skin surface dabbed clean to remove surface drug, and glecaprevir and pibrentasvir were extracted from skins. Cumulative amounts of glecaprevir and pibrentasvir delivered from each formulation to both skin and receiver compartments were also determined. To extract the drug from the skin, skin samples collected at 24 hours were cut into small pieces, where 0.5 ml of water was added to each sample. They were then homogenised for 15 minutes using a Tissue Lyser LT. Subsequently, 1 mL of methanol was added, and samples were homogenised again for another 15 minutes. Then they were transferred to the tubes followed by adding 3.5 mL of methanol. After a 30-min sonication, 100 μL of the samples were diluted to 1 mL by PBS buffer. The samples were vortexed and centrifuged, then analysed by HPLC.

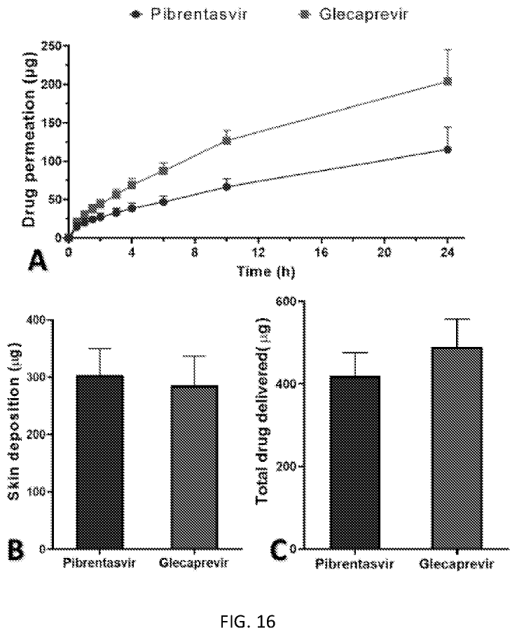

The data is summarised in FIG. 16. Over 24 h, 115.2±29 μg of pibrentasvir and 203.9±41.3 μg of glecaprevir were delivered to the receiver compartment of the Franz diffusion cells and 304±45.7 μg of pibrentasvir and 285.2±51.3 μg of glecaprevir were deposited into the skin over 24 h. This brings up the total amount delivered to the skin and receiver compartment over 24 hours to 419.2±56.6 μg of pibrentasvir and 489.1±68.6 μg of glecaprevir, or 23.4±3.2% of the pibrentasvir and 25.1±3.5% of the glecaprevir initially present in the microneedle arrays. The majority of the delivered glecaprevir and pibrentasvir remained in the skin, which is believed to be due to the high hydrophobicity of these compounds.

### Example 22: In Vivo Assessment of the Microneedles Compared to Intramuscularly and Intravenously Administered Glecaprevir and Pibrentasvir

Female Sprague-Dawley rats (total n=18), 8-10 weeks of age and possessing a mean weight of 266.3±28.3 g, were acclimatised for seven days prior to the experiment. Animals were separated into three cohorts; each was comprised of 6 rats. Every 3 rats were housed in a different cage. Each rat of the first cohort (rats 1-6) received four microneedle arrays with a total dose of 16 mg (8 mg of glecaprevir and 8 mg of pibrentasvir). The second cohort (rats 7-12) received intramuscular injections of glecaprevir and pibrentasvir. For each rat, 37.5 mg of the glecaprevir₃₀/pibrentasvir₃₀/Plasdone C15₃₀/AOT₁₀ formulation was dispersed in 300 μL of sterile water, then 150 μL of the solution was injected into each thigh of the rat. The dose given to each rat was approximately 60 mg/rat. The final cohort (rats 13-18) received 5 mg/kg of glecaprevir and pibrentasvir given as IV injections. The IV formulations were prepared by dissolving each drug in 5% v/v DMSO, 10% v/v PEG 400 in water diluted with 20% v/v hydroxy-B-cyclodextrin in water.

To minimise the interference of hair in application of the microneedle arrays, the dorsal hair of the rats from the first cohort (rats 1-6) was removed prior to the experiment. The bulk hair was shaved using an electric hair clipper and the remaining hair residuals were removed using depilatory hair removal cream. Rats were then left for a 24 hour period to allow the skin to recover and to ensure complete restoration of skin barrier function before affixing the microneedle arrays. The following day, rats were sedated using a gaseous anaesthetic gas (2-4% v/v isoflurane in oxygen), where microneedle arrays were affixed using firm thumb pressure onto a pinched section of skin on the back of the rats in cohort 1. Afterwards, Tegaderm™ film was placed on top of the microneedle arrays and kinesiology tape applied to keep them in place.

Blood samples were obtained using tail vein bleeding at predefined time intervals in preheparinised Eppendorf tubes. For the first and second cohorts groups, sampling took place at 1 h, 6 h, 24 h, 48 h, 72 h, 7 d, 14 d, 21 d, 28 d, 42 d, 49 d. For the third cohort, samples were taken at 5 min, 45 min, 2 h, 6 h, 24 h and 48 h. Samples were centrifuged at 14800 rpm for 4 mins to obtain plasma, which was then stored in a −80° C. freezer prior to their analysis.

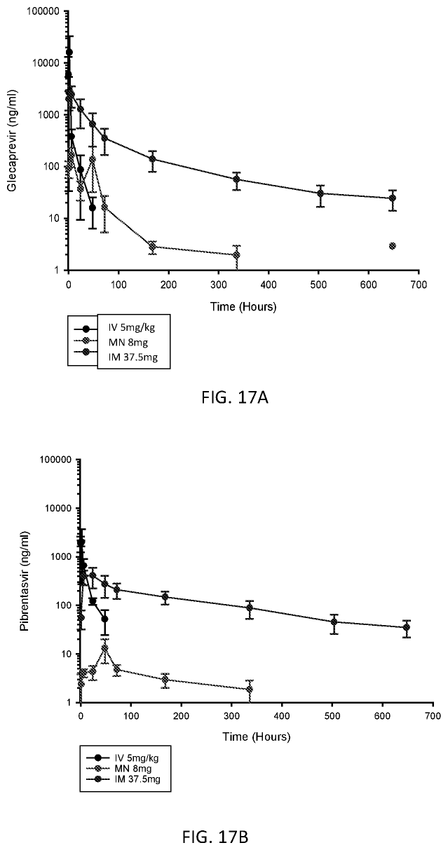

The results are summarised in FIGS. 17A and 7B and show that the plasma concentration of the intravenously administered (IV) glecaprevir and pibrentasvir subsided quickly within the 48 hour period that was monitored. Conversely, the glecaprevir and pibrentasvir that was administered transdermally (MN—via microneedle array) maintained their concentration for at least the first 72 hours, and that glecaprevir and pibrentasvir that was administered intramuscularly (IM) maintained their concentration for the duration of the experiment. NB the lower concentrations of glecaprevir and pibrentasvir detected for the microneedle arrays is due to the lower absolute quantity administered.

The foregoing experiments have shown that solid compositions comprising microparticles of glecaprevir and/or pibrentasvir may be formed through combination with certain first and second excipients. The solid compositions have also been shown to form stable aqueous dispersions on mixing with water. The solid compositions have also been reformulated into implantable rods, and microneedle arrays. Any of the solid compositions, aqueous dispersions, implantable rods, and microneedle arrays are expected to be therapeutically useful based on the known therapeutic uses of glecaprevir and pibrentasvir and the well-tolerated nature of the first and second excipients used. In addition, the form of the solid composition, aqueous dispersion, implantable rod, and microneedle array are expected to provide enhanced pharmacokinetic properties and therapeutic benefits when compared to conventional glecaprevir and pibrentasvir orally-dosed formulations. This is especially true of the aqueous dispersions and the injectable formulations, with the foregoing experiments demonstrating that the dispersion of the solid composition in water provides for aqueous dispersions having unprecedented concentrations of glecaprevir and/or pibrentasvir, thereby enabling the treatment of HCV infections with long acting injectable formulations. In particular, the formulations comprising microparticles of glecaprevir and/or pibrentasvir as described herein are expected to greatly improve the terminal half-life of the glecaprevir and/or pibrentasvir in vivo as compared to conventional aqueous formulations in which the glecaprevir and/or pibrentasvir are solubilised through addition of DMSO, when the formulations are delivered as intramuscular and/or subcutaneous injections. Conventional aqueous formulations of glecaprevir and pibrentasvir were found to have terminal half-lives of 4 hours and 10 hours respectively in adult male Wistar rats when injected intramuscularly. Following the teachings of this application, in particular the teachings that the release rate of glecaprevir and/or pibrentasvir from the depot is governed by their physicochemical properties and local physiological environment, the skilled person would expect the long acting injectable formulations as defined herein to be able to increase the terminal half-lives of glecaprevir and pibrentasvir to the extent that, in combination with the aqueous dispersions having unprecedented concentrations of glecaprevir and/or pibrentasvir in the aqueous dispersions while retaining syringeability, as demonstrated in the foregoing examples, they can provide a depot capable of providing a therapeutically effective concentration of each of glecaprevir and pibrentasvir for the duration of treatment (e.g. 3 weeks) through a single administration of the long acting injectable formulation. Subsequent experiments have demonstrated that the foregoing expectation is correct, with data showing that each of glecaprevir and pibrentasvir remains present in blood plasma in significant quantities after a period of 91-days (far in excess of the 3 weeks required) when administered as the long acting injectable, implantable rod, or microneedle array of the present invention.

1. A solid composition comprising microparticles of glecaprevir and/or pibrentasvir dispersed within a matrix comprising a first excipient and a second excipient,
wherein the first excipient is selected from polyvinylpyrrolidone (PVP), polyvinyl alcohol (PVA), polyethylene glycol (PEG), hydroxypropyl methylcellulose (HPMC), polyoxyethylene-polyoxypropylene block copolymer (poloxamer), polyethoxylated castor oil, PVA-PEG graft copolymer, lactose, and sucrose, and
wherein the second excipient is selected from dioctyl sodium sulfosuccinate (AOT), benzalkonium chloride, sodium deoxycholate (NDC), poloxamer, Polysorbate 20, Polysorbate 80, sorbitan monolaurate, polyethylene glycol (15)-hydroxystearate, and vit-E-PEG succinate.

2. The solid composition of claim 1 wherein the microparticles of glecaprevir and/or pibrentasvir are microparticles of pibrentasvir.

3. The solid composition of claim 2, wherein
the first excipient is selected from polyethoxylated castor oil, PVP, lactose, sucrose, poloxamer, and PEG; and
the second excipient is selected from benzalkonium chloride, AOT, poloxamer, Polysorbate 20, Polysorbate 80, and sorbitan monolaurate.

4. (canceled)

5. The solid composition of claim 2, wherein
the first excipient is selected from PVA, PEG, HPMC, poloxamer, and PVA-PEG graft copolymer; and
the second excipient is selected from AOT, Polysorbate 20, Polysorbate 80, NDC, polyethylene glycol (15)-hydroxystearate, and Vit-E-PEG succinate.

6. (canceled)

7. The solid composition of claim 1 wherein the microparticles of glecaprevir and/or pibrentasvir are microparticles of glecaprevir.

8. The solid composition of claim 7, wherein
the first excipient is selected from polyethoxylated castor oil, PVP, lactose, sucrose, poloxamer, and PEG; and
the second excipient is selected from benzalkonium chloride, AOT, poloxamer, Polysorbate 20, Polysorbate 80, and sorbitan monolaurate.

9. (canceled)

10. The solid composition of claim 7, wherein
the first excipient is selected from PVA, PEG, HPMC, poloxamer, and PVA-PEG graft copolymer; and
the second excipient is selected from AOT, Polysorbate 20, Polysorbate 80, NDC, polyethylene glycol (15)-hydroxystearate, and Vit-E-PEG succinate

11. (canceled)

12. The solid composition of claim 1, wherein the microparticles of glecaprevir and/or pibrentasvir are microparticles of glecaprevir and pibrentasvir.

13. The solid composition of claim 12, wherein
the first excipient is selected from polyethoxylated castor oil, PVP, lactose, sucrose, and poloxamer; and
the second excipient is selected from benzalkonium chloride, AOT, poloxamer, Polysorbate 20, Polysorbate 80, and sorbitan monolaurate.

14-15. (canceled)

16. The solid composition of claim 1, wherein the composition comprises:
40 to 80 wt % glecaprevir and/or pibrentasvir combined;
5 to 50 wt % of the first excipient; and
1 to 30 wt % of the second excipient.

17-18. (canceled)

19. The solid composition of claim 1, wherein the microparticles have at least one of:
(i) a particle diameter in the range of 10 to 2500 nm, preferably between 20 nm and 2000 nm, more preferably between 50 nm and 1500 nm, further preferably between 100 nm and 1000 nm, and most preferably between 150 and 450 nm; and
(ii) a polydispersity less than or equal to 0.8, preferably less than or equal to 0.6, more preferably less than or equal to 0.5.

20. (canceled)

21. A process for preparing a solid composition according claim 1, the process comprising:
(a) preparing an oil-in-water emulsion comprising:
an oil phase comprising glecaprevir and/or pibrentasvir; and
an aqueous phase comprising a first and second excipient, each as defined in claim 1; and
(b) removing the oil and water from the oil-in-water emulsion to form the solid composition; optionally wherein the step of removing the oil and water from the oil-in-water emulsion comprises spray drying or freeze-drying.

22. (canceled)

23. An aqueous dispersion comprising a plurality of microparticles of glecaprevir and/or pibrentasvir dispersed in an aqueous medium and stabilised by a mixture of a first excipient and a second excipient;
wherein the first excipient is selected from polyvinylpyrrolidone (PVP), polyvinyl alcohol (PVA), polyethylene glycol (PEG), hydroxypropyl methylcellulose (HPMC), polyoxyethylene-polyoxypropylene block copolymer (poloxamer), polyethoxylated castor oil, PVA-PEG graft copolymer, lactose, and sucrose,-and
wherein the second excipient is selected from sodium sulfosuccinate (AOT), benzalkonium chloride, dioctyl sodium deoxycholate (NDC), poloxamer, Polysorbate 20, Polysorbate 80, sorbitan monolaurate, polyethylene glycol (15)-hydroxystearate, and vit-E-PEG succinate; and
optionally wherein the glecaprevir and/or pibrentasvir is present in the aqueous dispersion at a concentration of 100 to 1000 mg/mL, preferably 200 to 900 mg/mL, more preferably 300 to 800 mg/mL, and most preferably 500 to 700 mg/mL.

24-25. (canceled)

26. A process for preparing an aqueous dispersion according to claim 23, the process comprising dispersing a solid composition according to any of claims 1 in an aqueous medium.

27. A pharmaceutical composition comprising the solid composition of claim 1, or the aqueous dispersion of any of claim 23 and, optionally, one or more further pharmaceutically acceptable excipients.

28. An injectable formulation comprising the solid composition of any claim 1, the aqueous dispersion of any of claim 23, or the pharmaceutical composition of claim 27, optionally wherein the injectable formulation is a subcutaneously or intramuscularly injectable formulation, optionally wherein the injectable formulation is suitable for provision in depot form.

29. (canceled)

30. A method of producing an implantable rod comprising the steps of compressing a solid composition according to claim 1 and heating the compressed solid composition for a period of time.

31. The method of claim 30, wherein in the method at least one of the following applies:
(i) the solid composition is compressed in a mould, optionally the mould being cylindrical in form;
(ii) the solid composition is heated to a temperature from 60 to 160° C., preferably from 80 to 140° C., more preferably from 100 to 120° C., most preferably about 110° C.;
(iii) the compression occurs under a reduced pressure atmosphere;
(iv) the heating step takes place for a period of from 1 minute to 30 minutes, preferably from 2 minutes to 25 minutes, more preferably from 5 minutes to 15minutes, most preferably about 10 minutes; and
(v) further comprises a step of cooling the rod, optionally the cooling taking place under a reduced pressure atmosphere.

32-36. (canceled)

37. An implantable rod comprising microparticles of glecaprevir and/or pibrentasvir dispersed within a monolith comprising a first excipient and a second excipient,
wherein the first excipient is selected from polyvinylpyrrolidone (PVP), polyvinyl alcohol (PVA), polyethylene glycol (PEG), hydroxypropyl methylcellulose (HPMC), polyoxyethylene-polyoxypropylene block copolymer (poloxamer), polyethoxylated castor oil, PVA-PEG graft copolymer, lactose, and sucrose, and
wherein the second excipient is selected from dioctyl sodium sulfosuccinate (AOT), benzalkonium chloride, sodium deoxycholate (NDC), poloxamer, Polysorbate 20, Polysorbate 80, sorbitan monolaurate, polyethylene glycol (15)-hydroxystearate, and vit-E-PEG succinate.

38. (canceled)

39. A method of producing a microneedle array comprising microneedles of a first composition arrayed on one face of a baseplate of a second composition, the method comprising the steps of:
a) dispersing a solid composition according to claim 1 and at least one structural polymer in a solvent to form a microneedle precursor dispersion;
b) placing the microneedle precursor dispersion into a mould;
c) compressing the microneedle precursor dispersion in the mould and then drying to form microneedles comprising the first composition;
d) adding a baseplate precursor solution into the mould;
e) compressing the baseplate precursor solution and then drying to form the baseplate of the second composition; and
f) releasing the microneedle array from the mould.

40. The method of claim 39, wherein in the method at least one of the following applies:
(i) steps b) and c) are repeated prior to steps d) to f);
(ii) the solvent is an aqueous solvent, such as water;
(iii) the at least one structural polymer is selected from PVA, PVP, and combinations thereof; and
(iv) the baseplate precursor solution comprises a base polymer selected from PVP and, optionally, one or more additives such as glycerol, dispersed in an aqueous solvent, such as water.

41-44. (canceled)

45. A microneedle array comprising microneedles of a first composition arrayed on one face of a baseplate of a second composition,
wherein the first composition comprises microparticles of glecaprevir and/or pibrentasvir dispersed within a monolith comprising:
a first excipient selected from polyvinylpyrrolidone (PVP), polyvinyl alcohol (PVA), polyethylene glycol (PEG), hydroxypropyl methylcellulose (HPMC), polyoxyethylene-polyoxypropylene block copolymer (poloxamer), polyethoxylated castor oil, PVA-PEG graft copolymer, lactose, and sucrose,
a second excipient selected from dioctyl sodium sulfosuccinate (AOT), benzalkonium chloride, sodium deoxycholate (NDC), poloxamer, Polysorbate 20, Polysorbate 80, sorbitan monolaurate, polyethylene glycol (15)-hydroxystearate, and vit-E-PEG succinate, and
at least one structural polymer.

46. (canceled)

47. The microneedle array of claim 45, wherein at least one of the following features is present:
(i) the at least one structural polymer is selected from PVA, PVP, and combinations thereof;
(ii) the second composition comprises a base polymer, such as PVP, and, optionally, one or more additives such as glycerol.

48-53. (canceled)

54. A method of treating and/or preventing HCV infection and/or Hepatitis C, the method comprising administering a therapeutically effective amount of a solid composition according to claim 1, an aqueous dispersion according to claim 23, a pharmaceutical composition according to claim 15, an injectable formulation according to claim 28, an implantable rod according to claim 36, or a microneedle array according to claim 45, to a patient suffering from or at risk of suffering from HCV infection and/or Hepatitis C.

55. The method of claim 54, wherein the concentration of glecaprevir and/or pibrentasvir within the aqueous dispersion, the pharmaceutical composition, or the injectable formulation is 100 to 1000 mg/mL, preferably 200 to 900 mg/mL, more preferably 300 to 800 mg/mL, and most preferably 500 to 700 mg/mL.

56. The method of claim 54, wherein the concentration of glecaprevir and/or pibrentasvir in the implantable rod is in the range of 40 to 80 wt %, preferably 50 to 70wt %, most preferably about 60 wt %.

57. The method of claim 54, wherein the microneedle array contains a mass of glecaprevir and/or pibrentasvir in the range of between 1 and 20 mg of glecaprevir and/or pibrentasvir, preferably between 2 and 10 mg, more preferably about 5 mg.

58. The method of any of claims 54 to 56claim 54, wherein the aqueous dispersion, the pharmaceutical composition, the injectable formulation, or the implantable rod forms a depot within the body of the patient, optionally wherein the depot maintains a therapeutically effective concentration of glecaprevir and/or pibrentasvir within the body of the patient for a period of at least two weeks, preferably at least three weeks, more preferably at least one month, and most preferably at least two months.

59. The method of claim 54-or claim 57, wherein the microneedle array gradually releases glecaprevir and/or pibrentasvir, optionally wherein the microneedle array maintains a therapeutically effective concentration of glecaprevir and/or pibrentasvir within the body of the patient for a period of at least 4 hours, preferably at least 6 hours, more preferably at least 12 hours, and most preferably at least 24 hours.

60. The method of claim 54, wherein the patient requires dosing with the aqueous dispersion, the pharmaceutical composition, the injectable formulation, or the implantable rod up to three times, preferably up to two times, most preferably only once, to maintain a therapeutically effective concentration of glecaprevir and/or pibrentasvir for the duration of the treatment.

61. The method of claim 54, wherein the patient requires dosing of the microneedle array up to six times per day, preferably up to four times per day, more preferably twice a day, and most preferably once a day, to maintain a therapeutically effective concentration of glecaprevir and/or pibrentasvir in the patient for the duration of the treatment.
